(19) United States
(12) Patent Application Publication
(10) Pub. No.: US 2024/0403770 A1
(43) Pub. Date: Dec. 5, 2024

(54) MODIFYING AN EVENT NOTIFICATION CONFIGURATION BASED ON INCIDENT RESOLUTION DATA

(71) Applicant: PagerDuty, Inc., San Francisco, CA (US)

(72) Inventors: Ophir Ronen, Seattle, WA (US); Justin David Kearns, Lexington, KY (US); Shawn Christopher Motley, Seattle, WA (US); Thomas Dziedzic, San Francisco, CA (US); Owen Howard Wilson, San Francisco, CA (US)

(21) Appl. No.: 18/800,634

(22) Filed: Aug. 12, 2024

Related U.S. Application Data
(63) Continuation of application No. 18/296,424, filed on Apr. 6, 2023.
Application No. 18/296,424 is a continuation of application No. 17/330,925, filed on May 26, 2021, now abandoned.
Application No. 17/330,925 is a continuation of application No. 15/967,411, filed on Apr. 30, 2018, now abandoned.
(60) Provisional application No. 62/554,498, filed on Sep. 5, 2017.

(51) Int. Cl. G06Q 10/0635; A61B 5/00; A61B 5/16; G06Q 10/0639; G06Q 10/067; G16H 10/60; G16H 20/30; G16H 40/20; G16H 50/30

(52) U.S. Cl. CPC G06Q 10/0635; A61B 5/0004; A61B 5/0022; A61B 5/165; A61B 5/4815; A61B 5/7264; A61B 5/7275; G06Q 10/06393; G06Q 10/067; G16H 10/60; G16H 20/30; G16H 50/30; A61B 2503/24; G16H 40/20

(57) ABSTRACT

An apparatus includes a memory and a processor. The processor is configured to execute instructions stored in the memory to determine interrupt events from a plurality of notification events generated by one or more of a plurality of computer systems; receive alert data indicating occurrence of monitored conditions in a managed information technology environment; assign, based on the alert data, incidents to the responder; modify, with respect to the responder, a notification configuration based on resolution data associated with the incidents and the interrupt events; and transmit a notification to the responder according to the modified notification configuration in response to assigning a new incident to the responder. The interrupt events are determined based on respective corresponding notification events being sent in respective manners designed to immediately alert a responder.

### CROSS-REFERENCE TO RELATED APPLICATIONS

This application is a continuation of U.S. patent application Ser. No. 18/296,424 filed on Apr. 6, 2023, which is a continuation of U.S. patent application Ser. No. 17/330,925 filed on May 26, 2021, which is a continuation of U.S. patent application Ser. No. 15/967,411 filed on Apr. 30, 2018, which claims priority to U.S. Provisional Patent Application Ser. No. 62/554,498 filed on Sep. 5, 2017, the entire disclosures of which are incorporated herein by reference in their entireties.

### TECHNICAL FIELD

The present invention relates generally to computer operations and more particularly, but not exclusively to providing real-time management of information technology operations and personnel scale in noisy, complex, distributed, heterogeneous, and dynamically changing environments.

### BACKGROUND

With the increase in complexity of distributed computing systems due to the growing demand and reliance on the Internet for business, the response to Operations Incidents is highly complex at all scales. An army of experts is often needed to deal with the complexity, pace of change, distributed nature of teams, speed of delivery, and the impact Incidents may have on the businesses. This may result in organizational complexity that may create the need for improved management to improve operational efficiency.

In some cases, an organization's operational pain may be inversely proportional to their operational maturity. Accordingly, high levels of operational pain may significantly and adversely impact overall performance of the business. For example, it may increase the risk of employees leaving the organization, or lead to pain experienced by customers due to errors made by poor and inefficient business practices.

The symptoms of operational pain include: responder on-call becomes a dreaded experience due to expected lack of sleep and exhaustion, and of course human error due to fatigue and information overload, degradation in quality of product delivered to customers, slow product velocity, low customer satisfaction, degraded corporate reputation, and loss of employees due to hostile work environment. Thus, it is with respect to these considerations and others that the present invention has been made.

### BRIEF DESCRIPTION OF THE DRAWINGS

Non-limiting and non-exhaustive embodiments of the present innovations are described with reference to the following drawings. In the drawings, like reference numerals refer to like parts throughout the various figures unless otherwise specified. For a better understanding of the described innovations, reference will be made to the following Detailed Description of Various Embodiments, which is to be read in association with the accompanying drawings, wherein:

FIG. 1 shows components of one embodiment of an environment in which embodiments of the invention may be practiced;

FIG. 2 shows one embodiment of a client computer that may be included in a system in accordance with at least one of the various embodiments;

FIG. 3 shows one embodiment of a network computer, in accordance with at least one of the various embodiments;

FIG. 4 illustrates a logical architecture of a system that provides operations health management in accordance with at least one of the various embodiments;

FIG. 5 illustrates a logical architecture of a system that provides operations health management in accordance with at least one of the various embodiments;

FIG. 6 illustrates a functional architecture of a system for providing model inputs to a scoring engine in accordance with one or more the various embodiments;

FIG. 7 illustrates a logical representation of an interactive report that enables organizations to manage their operations health in accordance with at least one of the various embodiments;

FIG. 8 illustrates a logical representation of an interactive report that enables organizations to manage their operations health in accordance with at least one of the various embodiments;

FIG. 9 illustrates a logical schema of a system that includes data structure for managing operations health in accordance with one or more of the various embodiments;

FIG. 10 illustrates an overview of a process that shows an example of how IT operations in an organization may impact an organization's operations health scores in accordance with one or more of the various embodiments;

FIG. 11 illustrates a logical representation of an interactive report that shows how an organization's operations health scores may impact business health of an organization in accordance with one or more of the various embodiments;

FIG. 12 illustrates a logical representation of an interactive report that shows how a portion of an organization's operations health score(s) key performance indicators (KPIs) have changed over a given time period in accordance with one or more of the various embodiments;

FIG. 13 illustrates a logical representation of an interactive report that includes a few example metrics and parameters that may provide various insights into an organization's operational health in accordance with one or more of the various embodiments;

FIG. 14 illustrates a logical representation of an interactive report that shows how additional insights into an organization's operations may be related to an organization's operations health scores in accordance with one or more of the various embodiments;

FIG. 15 illustrates a logical representation of an interactive report that shows contributing sources of pain for individual responders and services in accordance with one or more of the various embodiments;

FIG. 16 illustrates a logical representation of an interactive report that shows health score with the remediation trends related to contributing sources of pain for individual responders or services in accordance with one or more of the various embodiments;

FIG. 17 illustrates an overview flowchart for a process for operations health management in accordance with at least one the various embodiments;

FIG. 18 illustrates a flowchart for a process for operations health management in accordance with at least one the various embodiments;

FIG. 19 illustrates a flowchart for a process for operations health management in accordance with at least one the various embodiments; and

FIG. 20 illustrates a flowchart for a process for generating individual responder health profiles in accordance with at least one the various embodiments.

### DETAILED DESCRIPTION OF VARIOUS EMBODIMENTS

Various embodiments now will be described more fully hereinafter with reference to the accompanying drawings, which form a part hereof, and which show, by way of illustration, specific exemplary embodiments by which the invention may be practiced. The embodiments may, however, be embodied in many different forms and should not be construed as limited to the embodiments set forth herein; rather, these embodiments are provided so that this disclosure will be thorough and complete, and will fully convey the scope of the embodiments to those skilled in the art. Among other things, the various embodiments may be methods, systems, media or devices. Accordingly, the various embodiments may take the form of an entirely hardware embodiment, an entirely software embodiment or an embodiment combining software and hardware aspects. The following detailed description is, therefore, not to be taken in a limiting sense.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

For example embodiments, the following terms are also used herein according to the corresponding meaning, unless the context clearly dictates otherwise.

As used herein the term, “engine” refers to logic embodied in hardware or software instructions, which can be written in a programming language, such as C, C++, Objective-C, COBOL, Java™, PHP, Perl, JavaScript, Ruby, VBScript, Microsoft.NET™ languages such as C#, and/or the like. An engine may be compiled into executable programs or written in interpreted programming languages. Software engines may be callable from other engines or from themselves. Engines described herein refer to one or more logical modules that can be merged with other engines or applications, or can be divided into sub-engines. The engines can be stored in non-transitory computer-readable medium or computer storage devices and be stored on and executed by one or more general purpose computers, thus creating a special purpose computer configured to provide the engine.

The term “organization” as used herein refers to a business, a company, an association, an enterprise, a confederation, or the like.

The term “operations management system” as used herein is computer system that may be arranged to monitor, manage, and compare, the operations of one or more organizations. Operations management system may be arranged to accept various operations events that indicate events and/or incidents occurring in the managed organizations. Operations management systems may be arranged to manage several separate organizations at the same time. These separate organizations may be considered a community of organizations.

The terms “event,” “operations event” as used herein refer one or more outcomes, conditions, or occurrences that may be detected or observed by an operations management system. Operations management systems may be configured to monitor various types of events depending on needs of an industry and/or technology area. For example, information technology services may generate events in response to one or more conditions, such as, computers going offline, memory overutilization, CPU overutilization, storage quotas being met or exceeded, applications failing or otherwise becoming unavailable, networking problems (e.g., latency, excess traffic, unexpected lack of traffic, intrusion attempts, or the like), electrical problems (e.g., power outages, voltage fluctuations, or the like), customer service requests, or the like, or combination thereof.

Events or operations events may be provided to the operations management system using one or more messages, emails, telephone calls, library function calls, application programming interface (API) calls, including, any signals provided to an operations management system indicating that an event has occurred. One or more third party and/or external systems may be configured to generate event messages that are provided to the operations management system.

The term “resource” as used herein refers to a person or entity that may be responsible for responding to an event associated with a monitored application or service. For example, resources may be members of an information technology (IT) team providing support to employees of a company. Resources may be notified if an event they are responsible for handling at that time is encountered. In some embodiments, a scheduler application may be arranged to associate one or more resources with times that they are responsible for handling particular events (.e.g., times when they are on-call to maintain various IT services for a company). A resource that is determined to be responsible for handling a particular event may be referred to as a responsible resource. Responsible resources may be considered to be on-call and/or active during the period of time they are designated by the schedule to be available.

The term “incidents” as used herein may refer to a condition or state in the managed networking environments that requires some form of resolution by a user or automated service. Typically, incidents may be a failure or error that occurs in the operation of a managed network and/or computing environment. One or more events may be associated with one or more incidents. However, not all events are associated with incidents.

The terms “incident response” as used herein refer to the actions, resources, services, messages, notifications, alerts, events, or the like, related to resolving one or more incidents. Accordingly, services that may be impacted by a pending incident, may be added to the incident response associated with the incident. Likewise, resources responsible for supporting or maintaining the services may also be added to the incident response. Further, log entries, journal entries, notes, timelines, task lists, status information, or the like, may be part of an incident response.

The term “incident commander” as used herein refers to a user or resource that is responsible for administering an incident response.

The term “notification message,” or “notification event” as used herein refers to a communication provided by an incident management system to a message provider for delivery to one or more responsible resources or responders. A notification event may be used to inform one or more responsible resources that one or more operations event messages were received. For example, in at least one of the various embodiments, notification messages may be provided to the one or more responsible resources using SMS texts, MMS texts, email, Instant Messages, mobile device push notifications, HTTP requests, voice calls (telephone calls, Voice Over IP calls (VOIP), or the like), library function calls, API calls, URLs, audio alerts, haptic alerts, other signals, or the like, or combination thereof.

The term “message provider” as used herein refers to a first or third party service provider that communicates one or more notification messages to one or more responsible resources. Message providers may communicate with one or more types of technologies, such as, SMS texts, MMS texts, email, Instant Messages (IM), push notifications, HTTP requests, voice calls, library function calls, audio alerts, haptic alerts, any signals, or the like, or combination thereof. A notification system may employ one or more message providers to at least communicate notification messages to the one or more responsible resources.

The term “responder” as used herein refers to a resource that is a resource that is responsible for responding to one or more notification events.

The term “team” as used herein refers to one or more resources that may be jointly responsible for maintaining or supporting one or more services or system for an organization.

The term “service” as used herein refers to an organizational unit in an organization that provides one or more functional or operational systems that supply or provide various needs of an organization or an organization's customers.

The term “interrupt event” as used herein refers to a notification event that is of a type or format that requires a responders to actively address. Typically, interrupt events are notification events delivered using methods that either intentionally or unintentionally cause or require the receiving party (e.g., the responsible responder) to stop or interrupt what they are doing to review the interrupt event.

The terms “operations health sub-score,” or “health sub-score” as used herein may refer to a numerical values that represent one or more dimensions of the operations health of an organization. Sub-scores may be based on one or more health metrics or computations of health metrics. The health metrics associated with health sub-scores may represent continuous data or discrete data, including: measure of mean hour of day notifications are received; proportion of interrupting events during sleep hours; proportion of interrupt events during dinner hours; notification variation throughout the day (in hours); proportion of email notifications; proportion of interrupt events during weekends; proportion of days across time period with non-email notifications during sleep hours; proportion of days across time period with non-email notifications during dinner hours; proportion of days across time period with non-email notifications during evening hours; proportion of days across time period with non-email notifications during weekends; measure of successive days across time period with non-email notifications during sleep hours; measure of successive days across time period with non-email notifications during dinner hours; measure of successive days across time period with non-email notifications during evening hours; measure of successive days across time period with non-email notifications during weekend; measure of notification count with respect to distribution of notification counts across the organization; or the like, or combination thereof. Further, one of ordinary skill in the art will appreciate that there are other relevant metrics that may be generated, measured, or collected to use in sub-scores. It is in the interest of clarity and brevity that the description of additional metrics is omitted.

The term “operations health score” as used herein may be a value comprised of weighted values of the one or more health sub-scores. Different health sub-scores may have different weights depending on their contributions to the operations health of the organization or individuals in the organization. The operations health score may be arranged to be a single value that represents the operations health of an organization. Since the score may be generated consistently across multiple organizations the score may be useful for comparing an organization's operations health to other organizations. Likewise, operations health score associated with particular resources, such as, responders, teams, departments, or the like, may be used to compare operations health within the same organization.

The terms “operations health sub-score model,” or “health sub-score model” as used herein may refer to one or more data structures or computer readable instructions that may be generated to model one or more health sub-scores. A model may be generated based on various methods, such as, machine learning, linear regression, heuristics, other statistical modeling, or the like, or combination thereof. Accordingly, health metrics may be provided to one or more health sub-score models to evaluate operations health for organizations, parts of an organization, individuals, or the like.

The term “operations health model” as used herein may refer to one or more data structures or computer readable instructions that may be generated to model an organization's operations health. A operations health model may be generated based on various methods, such as, machine learning, linear regression, heuristics, other statistical modeling, or the like, or combination thereof. Also, operations health models may be composed of one or more sub-score models. Accordingly, operations health models may be used to evaluate operations health for organizations, parts of an organization, individuals, or the like.

The following briefly describes the embodiments of the invention in order to provide a basic understanding of some aspects of the invention. This brief description is not intended as an extensive overview. It is not intended to identify key or critical elements, or to delineate or otherwise narrow the scope. Its purpose is merely to present some concepts in a simplified form as a prelude to the more detailed description that is presented later.

Briefly stated, various embodiments are directed towards decreasing operational pain and increasing system efficiency through health monitoring and management. This may be accomplished through measuring, monitoring, reducing meaningful incident behavior across an organization and using it to inform necessary changes in the organizational operations to improve efficiency, or the like.

In one or more of the various embodiments, a resource management engine may be arranged to perform actions to actively manage the health of its IT operations. In one or more of the various embodiments, ergonomic data or metrics collected by a resource management engine may be used to intelligently inform proper system management decisions to increase human well-being across the organization's workforce to optimize overall system performance. In one or more of the various embodiments, health management may include using the information collected or provided by a resource management engine to recognize areas in the organization that need improvement or repair. In some embodiments, resource management engines may be arranged to automatically make one or more needed repairs. In one or more of the various embodiments, resource management engines may be arranged to execute a continuous feedback loop that may provide continuous overall system optimization.

In one or more of the various embodiments, resource management engines may be arranged to describe operations health in various contexts, including: services, teams, responders, or the like. In some embodiments, for services, resource management engines may be arranged to aggregate of the overall health as reported on Insights Service Graphs for defined time periods (e.g., interactive reports). For teams, resource management engines may be arranged to aggregate of the overall health as reported on Insights Team Graphs for defined time periods (e.g., interactive reports). And, for responders, resource management engines may be arranged to aggregate of the overall health as reported on Insights Responders Graphs for defined time periods (e.g., interactive reports).

In one or more of the various embodiments, resource management engines may be arranged to compile information from a variety of sources to employ for providing operations health scores. In one or more of the various embodiments, operational health may be analyzed across various parameters, including, sleep interruptions, dinner interruptions, successive days of notifications, push vs. email notifications, weekday vs weekends, or the like, or combination thereof.

In one or more of the various embodiments, one or more resource management engines may be instantiated to various perform actions associated with operations health management.

In one or more of the various embodiments, the one or more resource management engines may be instantiated to perform actions, including, employing a plurality of notification events to determine one or more interrupt events such that the one or more interrupt events require one or more responders to suspend a current activity to timely determine one or more responses to the one or more interrupt events.

In one or more of the various embodiments, the one or more resource management engines may be instantiated to perform actions, including, determining one or more sub-scores in real time based on one or more metrics being provided as input to one or more provided sub-score models, wherein the one or more metrics are associated with the one or more interrupt events. In one or more of the various embodiments, the one or more metrics may include one or more values that represent one or more of a measure of mean hour of day notifications are received, a proportion of interrupting events during sleep hours, a proportion of interrupt events during dinner hours, a measure of notification variation throughout a time period, a proportion of email notifications, a proportion of interrupt events during weekends, or the like, such that the one or more values may be provided from continuous data or discrete data.

In one or more of the various embodiments, the one or more resource management engines may be instantiated to perform actions, including, providing an operations score that is associated with a probability of an occurrence of one or more adverse outcomes based on the one or more sub-scores being provided as input to an operations model.

In one or more of the various embodiments, the one or more resource management engines may be instantiated to perform actions, including, providing one or more visualizations that represent one or more distributions of the one or more interrupt events.

In one or more of the various embodiments, the one or more resource management engines may be instantiated to perform actions, including, providing one or more reports that illustrate one or more increases in resources associated with the one or more distributions and the operations score.

In one or more of the various embodiments, the one or more resource management engines may be instantiated to perform actions, including, providing one or more individual sub-scores for the one or more responders based on the one or more interrupt events; providing an individual profile for the one or more responders based on a mapping of the one or more adverse outcomes to the one or more individual sub-scores; and predicting each of the one or more responders that have a high probability of being at risk for an adverse outcome based on the individual profile.

In one or more of the various embodiments, the one or more resource management engines may perform further actions, including, predicting an operations score based on the one or more metrics and the one or more sub-score models and the operations model.

In one or more of the various embodiments, the one or more resource management engines may perform further actions, including, providing operations score for one or more of teams, services, or departments that are associated with two or more responders.

In one or more of the various embodiments, the one or more analysis engines may be instantiated to perform actions, including, comparing the operations score to one or more other operations scores, wherein the comparison of operations scores reduces an amount of computing resources required to predict in real time the one or more adverse outcomes.

In one or more of the various embodiments, the one or more analysis engines may be instantiated to perform actions, including, updating one or more coefficients of the one or more sub-score models when a result of the comparison exceeds a threshold.

In one or more of the various embodiments, the one or more analysis engines may be instantiated to perform actions, including, recommending one or more actions to decrease the probability of the occurrence of the one or more adverse outcomes based on the comparison, wherein the one or more actions are provided in a report to a user. In one or more of the various embodiments, the one or more adverse outcomes include one or more of the one or more responders leaving the organization, an increase in production errors, reduced responder productivity, or the like.

In one or more of the various embodiments, a modeling engine may be instantiated to performs actions, including, providing the one or more sub-score models based on the metrics; and providing the operations model based on the one or more sub-score models.

### Illustrated Operating Environment

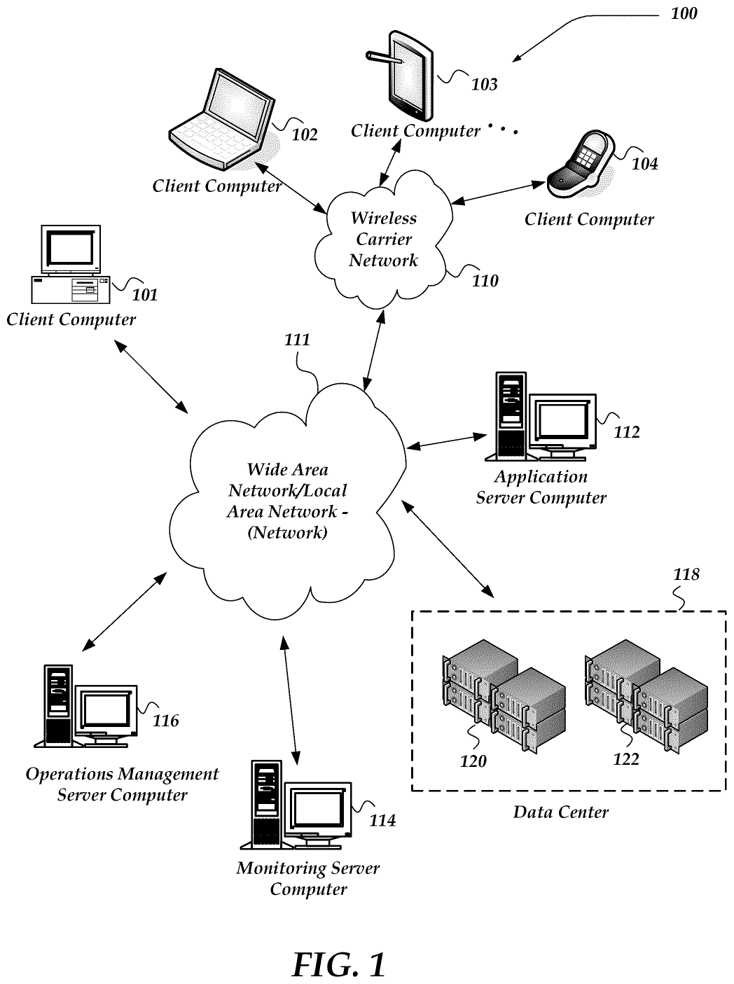

FIG. 1 shows components of one embodiment of an environment in which the invention may be practiced. Not all the components may be required to practice various embodiments, and variations in the arrangement and type of the components may be made. As shown, system 100 of FIG. 1 includes local area networks (“LANs”)/wide area networks (“WANs”)-(network) 111, wireless network 110, client computers 101-104, application server 112, monitoring server 114, and operations management server computer 116.

Generally, client computers 102-104 may include virtually any portable computing device capable of receiving and sending a message over a network, such as network 111, wireless network 110, or the like. Client computers 102-104 may also be described generally as client computers that are configured to be portable. Thus, client computers 102-104 may include virtually any portable computing device capable of connecting to another computing device and receiving information. Such devices include portable devices such as, cellular telephones, smart phones, display pagers, radio frequency (RF) devices, infrared (IR) devices, Personal Digital Assistants (PDA's), handheld computers, laptop computers, wearable computers, tablet computers, integrated devices combining one or more of the preceding devices, or the like. Likewise, client computers 102-104 may include Internet-of-Things (IoT) devices as well. Accordingly, client computers 102-104 typically range widely in terms of capabilities and features. For example, a cell phone may have a numeric keypad and a few lines of monochrome Liquid Crystal Display (LCD) on which only text may be displayed. In another example, a mobile device may have a touch sensitive screen, a stylus, and several lines of color LCD in which both text and graphics may be displayed.

Client computer 101 may include virtually any computing device capable of communicating over a network to send and receive information, including messaging, performing various online actions, or the like. The set of such devices may include devices that typically connect using a wired or wireless communications medium such as personal computers, multiprocessor systems, microprocessor-based or programmable consumer electronics, network Personal Computers (PCs), or the like. In one embodiment, at least some of client computers 102-104 may operate over wired and/or wireless network. Today, many of these devices include a capability to access and/or otherwise communicate over a network such as network 111 and/or even wireless network 110. Moreover, client computers 102-104 may access various computing applications, including a browser, or other web-based application.

In one embodiment, one or more of client computers 101-104 may be configured to operate within a business or other entity to perform a variety of services for the business or other entity. For example, client computers 101-104 may be configured to operate as a web server, an accounting server, a production server, an inventory server, or the like. However, client computers 101-104 are not constrained to these services and may also be employed, for example, as an end-user computing node, in other embodiments. Further, it should be recognized that more or less client computers may be included within a system such as described herein, and embodiments are therefore not constrained by the number or type of client computers employed.

A web-enabled client computer may include a browser application that is configured to receive and to send web pages, web-based messages, or the like. The browser application may be configured to receive and display graphics, text, multimedia, or the like, employing virtually any web-based language, including a wireless application protocol messages (WAP), or the like. In one embodiment, the browser application is enabled to employ Handheld Device Markup Language (HDML), Wireless Markup Language (WML), WMLScript, JavaScript, Standard Generalized Markup Language (SGML), HyperText Markup Language (HTML), extensible Markup Language (XML), HTML5, or the like, to display and send a message. In one embodiment, a user of the client computer may employ the browser application to perform various actions over a network.

Client computers 101-104 also may include at least one other client application that is configured to receive and/or send data, operations information, between another computing device. The client application may include a capability to provide requests and/or receive data relating to managing, operating, or configuring the operations management server computer 116.

Wireless network 110 is configured to couple client computers 102-104 and its components with network 111. Wireless network 110 may include any of a variety of wireless sub-networks that may further overlay stand-alone ad-hoc networks, or the like, to provide an infrastructure-oriented connection for client computers 102-104. Such sub-networks may include mesh networks, Wireless LAN (WLAN) networks, cellular networks, or the like.

Wireless network 110 may further include an autonomous system of terminals, gateways, routers, or the like connected by wireless radio links, or the like. These connectors may be configured to move freely and randomly and organize themselves arbitrarily, such that the topology of wireless network 110 may change rapidly.

Wireless network 110 may further employ a plurality of access technologies including 2nd (2G), 3rd (3G), 4th (4G), 5th (5G) generation radio access for cellular systems, WLAN, Wireless Router (WR) mesh, or the like. Access technologies such as 2G, 3G, 4G, and future access networks may enable wide area coverage for mobile devices, such as client computers 102-104 with various degrees of mobility. For example, wireless network 110 may enable a radio connection through a radio network access such as Global System for Mobil communication (GSM), General Packet Radio Services (GPRS), Enhanced Data GSM Environment (EDGE), Wideband Code Division Multiple Access (WCDMA), or the like. In essence, wireless network 110 may include virtually any wireless communication mechanism by which information may travel between client computers 102-104 and another computing device, network, or the like.

Network 111 is configured to couple network devices with other computing devices, including, schedule manager server 116, monitoring server 114, application server 112, client computer(s) 101, and through wireless network 110 to client computers 102-104. Network 111 is enabled to employ any form of computer readable media for communicating information from one electronic device to another. Also, network 111 can include the internet in addition to local area networks (LANs), wide area networks (WANs), direct connections, such as through a universal serial bus (USB) port, other forms of computer-readable media, or any combination thereof. On an interconnected set of LANs, including those based on differing architectures and protocols, a router acts as a link between LANs, enabling messages to be sent from one to another. In addition, communication links within LANs typically include twisted wire pair or coaxial cable, while communication links between networks may utilize analog telephone lines, full or fractional dedicated digital lines including T1, T2, T3, and T4, Integrated Services Digital Networks (ISDNs), Digital Subscriber Lines (DSLs), wireless links including satellite links, or other communications links known to those skilled in the art. For example, various Internet Protocols (IP), Open Systems Interconnection (OSI) architectures, and/or other communication protocols, architectures, models, and/or standards, may also be employed within network 111 and wireless network 110. Furthermore, remote computers and other related electronic devices could be remotely connected to either LANs or WANs via a modem and temporary telephone link. In essence, network 111 includes any communication method by which information may travel between computing devices.

Additionally, communication media typically embodies computer-readable instructions, data structures, program modules, or other transport mechanism and includes any information delivery media. By way of example, communication media includes wired media such as twisted pair, coaxial cable, fiber optics, wave guides, and other wired media and wireless media such as acoustic, RF, infrared, and other wireless media. Such communication media is distinct from, however, computer-readable devices described in more detail below.

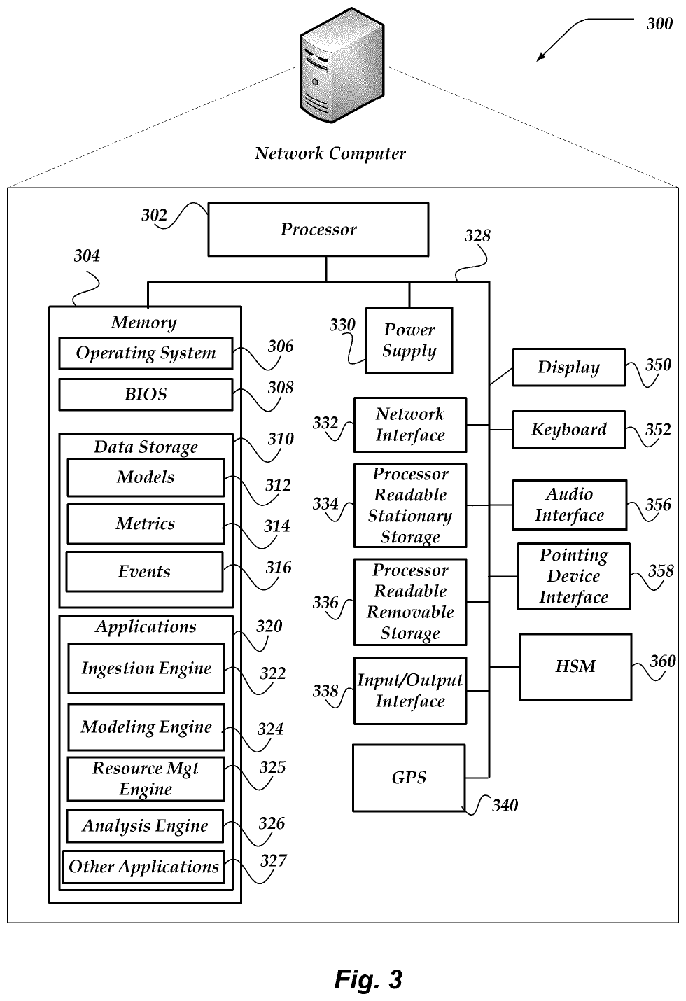

Operations management server computer 116 may include virtually any network computer usable to provide computer operations management services, such as network computer 300 of FIG. 3. In one embodiment, operations management server computer 116 employs various techniques for managing the operations of computer operations, networking performance, customer service, customer support, resource schedules and notification policies, event management, operations health management, or the like. Also, operations management server computer 116 may be arranged to interface/integrate with one or more external systems such as telephony carriers, email systems, web services, or the like, to perform computer operations management. Further, operations management server computer 116 may obtain various events and/or performance metrics collected by other systems, such as, monitoring server computer 114.

In at least one of the various embodiments, monitoring server computer 114 represents various computers that may be arranged to monitor the performance of computer operations for an entity (e.g., company or enterprise). For example, monitoring server computer 114 may be arranged to monitor whether applications/systems are operational, network performance, trouble tickets and/or their resolution, or the like. In some embodiments, one or more of the functions of monitoring server computer 114 may be performed by operations management server computer 116.

Devices that may operate as operations management server computer 116 include various network computers, including, but not limited to personal computers, desktop computers, multiprocessor systems, microprocessor-based or programmable consumer electronics, network PCs, server devices, network appliances, or the like. It should be noted that while operations management server computer 116 is illustrated as a single network computer, the invention is not so limited. Thus, operations management server computer 116 may represent a plurality of network computers. For example, in one embodiment, operations management server computer 116 may be distributed over a plurality of network computers and/or implemented using cloud architecture.

Moreover, operations management server computer 116 is not limited to a particular configuration. Thus, operations management server computer 116 may operate using a master/slave approach over a plurality of network computers, within a cluster, a peer-to-peer architecture, and/or any of a variety of other architectures.

In some embodiments, one or more data centers, such as, data center 118 may be communicatively coupled to network 111 and/or network 108. In at least one of the various embodiments, data center 118 may be a portion of a private data center, public data center, public cloud environment, or private cloud environment. In some embodiments, data center 118 may be a server room/data center that is physically under the control of an organization. Data center 118 may include one or more enclosures of network computers, such as, enclosure 120 and enclosure 122.

Enclosure 120 and enclosure 122 may be enclosures (e.g., racks, cabinets, or the like) of network computers and/or blade servers in data center 118. In some embodiments, enclosure 120 and enclosure 122 may be arranged to include one or more network computers arranged to operate as operations management server computers, monitoring server computers (e.g., operations management service computer 116, monitoring server computer 114, or the like), storage computers, or the like, or combination thereof. Further, one or more cloud instances may be operative on one or more network computers included in enclosure 120 and enclosure 122.

Also, data center 118 may include one or more public or private cloud networks. Accordingly, data center 118 may comprise multiple physical network computers, interconnected by one or more networks, such as, networks similar to and/or including network 108 and/or wireless network 110. Data center 118 may enable and/or provide one or more cloud instances (not shown). The number and composition of cloud instances may be vary depending on the demands of individual users, cloud network arrangement, operational loads, performance considerations, application needs, operational policy, or the like. In at least one of the various embodiments, data center 118 may be arranged as a hybrid network that includes a combination of hardware resources, private cloud resources, public cloud resources, or the like.

Thus, operations management server computer 116 is not to be construed as being limited to a single environment, and other configurations, and architectures are also contemplated. Operations management server computer 116 may employ processes such as described below in conjunction with at some of the figures discussed below to perform at least some of its actions.

### Illustrative Client Computer

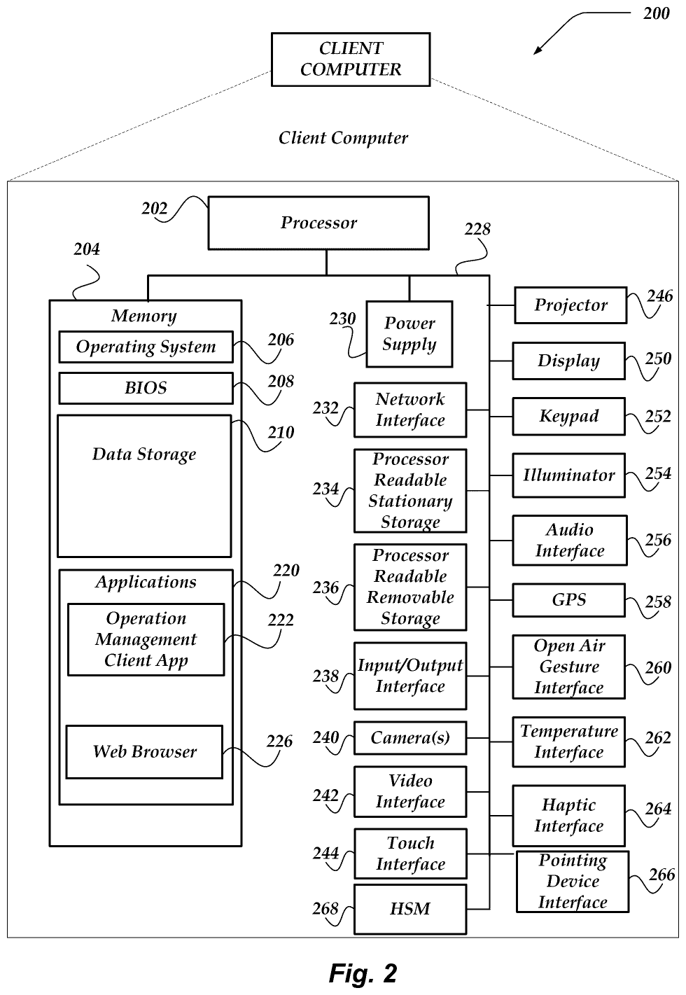

FIG. 2 shows one embodiment of client computer 200 that may include many more or less components than those shown. Client computer 200 may represent, for example, at least one embodiment of mobile computers or client computers shown in FIG. 1.

Client computer 200 may include processor 202 in communication with memory 204 via bus 228. Client computer 200 may also include power supply 230, network interface 232, audio interface 256, display 250, keypad 252, illuminator 254, video interface 242, input/output interface 238, haptic interface 264, global positioning systems (GPS) receiver 258, open air gesture interface 260, temperature interface 262, camera(s) 240, projector 246, pointing device interface 266, processor-readable stationary storage device 234, and processor-readable removable storage device 236. Client computer 200 may optionally communicate with a base station (not shown), or directly with another computer. And in one embodiment, although not shown, a gyroscope may be employed within client computer 200 to measuring or maintaining an orientation of client computer 200.

Power supply 230 may provide power to client computer 200. A rechargeable or non-rechargeable battery may be used to provide power. The power may also be provided by an external power source, such as an AC adapter or a powered docking cradle that supplements or recharges the battery.

Network interface 232 includes circuitry for coupling client computer 200 to one or more networks, and is constructed for use with one or more communication protocols and technologies including, but not limited to, protocols and technologies that implement any portion of the OSI model for mobile communication (GSM), CDMA, time division multiple access (TDMA), UDP, TCP/IP, SMS, MMS, GPRS, WAP, UWB, WiMax, SIP/RTP, GPRS, EDGE, WCDMA, LTE, UMTS, OFDM, CDMA2000, EV-DO, HSDPA, or any of a variety of other wireless communication protocols. Network interface 232 is sometimes known as a transceiver, transceiving device, or network interface card (NIC).

Audio interface 256 may be arranged to produce and receive audio signals such as the sound of a human voice. For example, audio interface 256 may be coupled to a speaker and microphone (not shown) to enable telecommunication with others or generate an audio acknowledgement for some action. A microphone in audio interface 256 can also be used for input to or control of client computer 200, e.g., using voice recognition, detecting touch based on sound, and the like.

Display 250 may be a liquid crystal display (LCD), gas plasma, electronic ink, light emitting diode (LED), Organic LED (OLED) or any other type of light reflective or light transmissive display that can be used with a computer. Display 250 may also include a touch interface 244 arranged to receive input from an object such as a stylus or a digit from a human hand, and may use resistive, capacitive, surface acoustic wave (SAW), infrared, radar, or other technologies to sense touch or gestures.

Projector 246 may be a remote handheld projector or an integrated projector that is capable of projecting an image on a remote wall or any other reflective object such as a remote screen.

Video interface 242 may be arranged to capture video images, such as a still photo, a video segment, an infrared video, or the like. For example, video interface 242 may be coupled to a digital video camera, a web-camera, or the like. Video interface 242 may comprise a lens, an image sensor, and other electronics. Image sensors may include a complementary metal-oxide-semiconductor (CMOS) integrated circuit, charge-coupled device (CCD), or any other integrated circuit for sensing light.

Keypad 252 may comprise any input device arranged to receive input from a user. For example, keypad 252 may include a push button numeric dial, or a keyboard. Keypad 252 may also include command buttons that are associated with selecting and sending images.

Illuminator 254 may provide a status indication or provide light. Illuminator 254 may remain active for specific periods of time or in response to event messages. For example, when illuminator 254 is active, it may backlight the buttons on keypad 252 and stay on while the client computer is powered. Also, illuminator 254 may backlight these buttons in various patterns when particular actions are performed, such as dialing another client computer. Illuminator 254 may also cause light sources positioned within a transparent or translucent case of the client computer to illuminate in response to actions.

Further, client computer 200 may also comprise hardware security module (HSM) 268 for providing additional tamper resistant safeguards for generating, storing or using security/cryptographic information such as, keys, digital certificates, passwords, passphrases, two-factor authentication information, or the like. In some embodiments, hardware security module may be employed to support one or more standard public key infrastructures (PKI), and may be employed to generate, manage, or store keys pairs, or the like. In some embodiments, HSM 268 may be a stand-alone computer, in other cases, HSM 268 may be arranged as a hardware card that may be added to a client computer.

Client computer 200 may also comprise input/output interface 238 for communicating with external peripheral devices or other computers such as other client computers and network computers. The peripheral devices may include an audio headset, display screen glasses, remote speaker system, remote speaker and microphone system, and the like. Input/output interface 238 can utilize one or more technologies, such as Universal Serial Bus (USB), Infrared, WiFi, WiMax, Bluetooth™, and the like.

Input/output interface 238 may also include one or more sensors for determining geolocation information (e.g., GPS), monitoring electrical power conditions (e.g., voltage sensors, current sensors, frequency sensors, and so on), monitoring weather (e.g., thermostats, barometers, anemometers, humidity detectors, precipitation scales, or the like), or the like. Sensors may be one or more hardware sensors that collect or measure data that is external to client computer 200.

Haptic interface 264 may be arranged to provide tactile feedback to a user of the client computer. For example, the haptic interface 264 may be employed to vibrate client computer 200 in a particular way when another user of a computer is calling. Temperature interface 262 may be used to provide a temperature measurement input or a temperature changing output to a user of client computer 200. Open air gesture interface 260 may sense physical gestures of a user of client computer 200, for example, by using single or stereo video cameras, radar, a gyroscopic sensor inside a computer held or worn by the user, or the like. Camera 240 may be used to track physical eye movements of a user of client computer 200.

GPS transceiver 258 can determine the physical coordinates of client computer 200 on the surface of the Earth, which typically outputs a location as latitude and longitude values. GPS transceiver 258 can also employ other geo-positioning mechanisms, including, but not limited to, triangulation, assisted GPS (AGPS), Enhanced Observed Time Difference (E-OTD), Cell Identifier (CI), Service Area Identifier (SAI), Enhanced Timing Advance (ETA), Base Station Subsystem (BSS), or the like, to further determine the physical location of client computer 200 on the surface of the Earth. It is understood that under different conditions, GPS transceiver 258 can determine a physical location for client computer 200. In at least one embodiment, however, client computer 200 may, through other components, provide other information that may be employed to determine a physical location of the client computer, including for example, a Media Access Control (MAC) address, IP address, and the like.

Human interface components can be peripheral devices that are physically separate from client computer 200, allowing for remote input or output to client computer 200. For example, information routed as described here through human interface components such as display 250 or keyboard 252 can instead be routed through network interface 232 to appropriate human interface components located remotely. Examples of human interface peripheral components that may be remote include, but are not limited to, audio devices, pointing devices, keypads, displays, cameras, projectors, and the like. These peripheral components may communicate over a Pico Network such as Bluetooth™, Bluetooth LE, Zigbee™ and the like. One non-limiting example of a client computer with such peripheral human interface components is a wearable computer, which might include a remote pico projector along with one or more cameras that remotely communicate with a separately located client computer to sense a user's gestures toward portions of an image projected by the pico projector onto a reflected surface such as a wall or the user's hand.

A client computer may include web browser application 226 that is configured to receive and to send web pages, web-based messages, graphics, text, multimedia, and the like. The client computer's browser application may employ virtually any programming language, including a wireless application protocol messages (WAP), and the like. In at least one embodiment, the browser application is enabled to employ Handheld Device Markup Language (HDML), Wireless Markup Language (WML), WMLScript, JavaScript, Standard Generalized Markup Language (SGML), HyperText Markup Language (HTML), extensible Markup Language (XML), HTML5, and the like.

Memory 204 may include RAM, ROM, or other types of memory. Memory 204 illustrates an example of computer-readable storage media (devices) for storage of information such as computer-readable instructions, data structures, program modules or other data. Memory 204 may store BIOS 208 for controlling low-level operation of client computer 200. The memory may also store operating system 206 for controlling the operation of client computer 200. It will be appreciated that this component may include a general-purpose operating system such as a version of UNIX, or LINUX™, or a specialized client computer communication operating system such as Windows Phone™, or IOS® operating system. The operating system may include, or interface with a Java virtual machine module that enables control of hardware components or operating system operations via Java application programs.

Memory 204 may further include one or more data storage 210, which can be utilized by client computer 200 to store, among other things, applications 220 or other data. For example, data storage 210 may also be employed to store information that describes various capabilities of client computer 200. The information may then be provided to another device or computer based on any of a variety of methods, including being sent as part of a header during a communication, sent upon request, or the like. Data storage 210 may also be employed to store social networking information including address books, buddy lists, aliases, user profile information, or the like. Data storage 210 may further include program code, data, algorithms, and the like, for use by a processor, such as processor 202 to execute and perform actions. In one embodiment, at least some of data storage 210 might also be stored on another component of client computer 200, including, but not limited to, non-transitory processor-readable removable storage device 236, processor-readable stationary storage device 234, or even external to the client computer.

Applications 220 may include computer executable instructions which, when executed by client computer 200, transmit, receive, or otherwise process instructions and data. Applications 220 may include, for example, operations management client application 222. In at least one of the various embodiments, operations management client application 222 may be used to exchange communications to and from operations management server computer 116, monitoring server computer 114, application server computer 112, or the like. Exchanged communications may include, but are not limited to, queries, searches, messages, notification messages, event messages, alerts, performance metrics, responder operations health score information, team operations health score information, services operations health score information, log data, API calls, or the like, combination thereof.

Other examples of application programs include calendars, search programs, email client applications, IM applications, SMS applications, Voice Over Internet Protocol (VOIP) applications, contact managers, task managers, transcoders, database programs, word processing programs, security applications, spreadsheet programs, games, search programs, and so forth.

Additionally, in one or more embodiments (not shown in the figures), client computer 200 may include an embedded logic hardware device instead of a CPU, such as, an Application Specific Integrated Circuit (ASIC), Field Programmable Gate Array (FPGA), Programmable Array Logic (PAL), or the like, or combination thereof. The embedded logic hardware device may directly execute its embedded logic to perform actions. Also, in one or more embodiments (not shown in the figures), client computer 200 may include a hardware microcontroller instead of a CPU. In at least one embodiment, the microcontroller may directly execute its own embedded logic to perform actions and access its own internal memory and its own external Input and Output Interfaces (e.g., hardware pins or wireless transceivers) to perform actions, such as System On a Chip (SOC), or the like.

### Illustrative Network Computer

FIG. 3 shows one embodiment of network computer 300 that may be included in a system implementing at least one of the various embodiments. Network computer 300 may include many more or less components than those shown in FIG. 3. However, the components shown are sufficient to disclose an illustrative embodiment for practicing these innovations. Network computer 300 may represent, for example, one embodiment of at least one of operations management server computer 116, monitoring server computer(s) 114, or application server computer(s) 112 of FIG. 1. Further, in some embodiments, network computer 300 may represent one or more network computers included in a data center, such as, data center 118, enclosure 120, enclosure 122, or the like.

As shown in the figure, network computer 300 includes a processor 302 in communication with a memory 304 via a bus 328. Network computer 300 also includes a power supply 330, network interface 332, audio interface 356, display 350, keyboard 352, input/output interface 338, processor-readable stationary storage device 334, and processor-readable removable storage device 336. Power supply 330 provides power to network computer 300.

Network interface 332 includes circuitry for coupling network computer 300 to one or more networks, and is constructed for use with one or more communication protocols and technologies including, but not limited to, protocols and technologies that implement any portion of the Open Systems Interconnection model (OSI model), global system for mobile communication (GSM), code division multiple access (CDMA), time division multiple access (TDMA), user datagram protocol (UDP), transmission control protocol/Internet protocol (TCP/IP), Short Message Service (SMS), Multimedia Messaging Service (MMS), general packet radio service (GPRS), WAP, ultra wide band (UWB), IEEE 802.16 Worldwide Interoperability for Microwave Access (WiMax), Session Initiation Protocol/Real-time Transport Protocol (SIP/RTP), or any of a variety of other wired and wireless communication protocols. Network interface 332 is sometimes known as a transceiver, transceiving device, or network interface card (NIC). Network computer 300 may optionally communicate with a base station (not shown), or directly with another computer.

Audio interface 356 is arranged to produce and receive audio signals such as the sound of a human voice. For example, audio interface 356 may be coupled to a speaker and microphone (not shown) to enable telecommunication with others or generate an audio acknowledgement for some action. A microphone in audio interface 356 can also be used for input to or control of network computer 300, for example, using voice recognition.

Display 350 may be a liquid crystal display (LCD), gas plasma, electronic ink, light emitting diode (LED), Organic LED (OLED) or any other type of light reflective or light transmissive display that can be used with a computer. Display 350 may be a handheld projector or pico projector capable of projecting an image on a wall or other object.

Network computer 300 may also comprise input/output interface 338 for communicating with external devices or computers not shown in FIG. 3. Input/output interface 338 can utilize one or more wired or wireless communication technologies, such as USB™, Firewire™, WiFi, WiMax, Thunderbolt™, Infrared, Bluetooth™, Zigbee™, serial port, parallel port, and the like.

Also, input/output interface 338 may also include one or more sensors for determining geolocation information (e.g., GPS), monitoring electrical power conditions (e.g., voltage sensors, current sensors, frequency sensors, and so on), monitoring weather (e.g., thermostats, barometers, anemometers, humidity detectors, precipitation scales, or the like), or the like. Sensors may be one or more hardware sensors that collect or measure data that is external to network computer 300. Human interface components can be physically separate from network computer 300, allowing for remote input or output to network computer 300. For example, information routed as described here through human interface components such as display 350 or keyboard 352 can instead be routed through the network interface 332 to appropriate human interface components located elsewhere on the network. Human interface components include any component that allows the computer to take input from, or send output to, a human user of a computer. Accordingly, pointing devices such as mice, styluses, track balls, or the like, may communicate through pointing device interface 358 to receive user input.

GPS transceiver 340 can determine the physical coordinates of network computer 300 on the surface of the Earth, which typically outputs a location as latitude and longitude values. GPS transceiver 340 can also employ other geo-positioning mechanisms, including, but not limited to, triangulation, assisted GPS (AGPS), Enhanced Observed Time Difference (E-OTD), Cell Identifier (CI), Service Area Identifier (SAI), Enhanced Timing Advance (ETA), Base Station Subsystem (BSS), or the like, to further determine the physical location of network computer 300 on the surface of the Earth. It is understood that under different conditions, GPS transceiver 340 can determine a physical location for network computer 300. In at least one embodiment, however, network computer 300 may, through other components, provide other information that may be employed to determine a physical location of the client computer, including for example, a Media Access Control (MAC) address, IP address, and the like.

Memory 304 may include Random Access Memory (RAM), Read-Only Memory (ROM), or other types of memory. Memory 304 illustrates an example of computer-readable storage media (devices) for storage of information such as computer-readable instructions, data structures, program modules or other data. Memory 304 stores a basic input/output system (BIOS) 308 for controlling low-level operation of network computer 300. The memory also stores an operating system 306 for controlling the operation of network computer 300. It will be appreciated that this component may include a general-purpose operating system such as a version of UNIX, or LINUX™, or a specialized operating system such as Microsoft Corporation's Windows® operating system, or the Apple Corporation's IOS® operating system. The operating system may include, or interface with a Java virtual machine module that enables control of hardware components or operating system operations via Java application programs. Likewise, other runtime environments may be included.

Memory 304 may further include one or more data storage 310, which can be utilized by network computer 300 to store, among other things, applications 320 or other data. For example, data storage 310 may also be employed to store information that describes various capabilities of network computer 300. The information may then be provided to another device or computer based on any of a variety of methods, including being sent as part of a header during a communication, sent upon request, or the like. Data storage 410 may also be employed to store social networking information including address books, buddy lists, aliases, user profile information, or the like. Data storage 310 may further include program code, instructions, data, algorithms, and the like, for use by a processor, such as processor 302 to execute and perform actions such as those actions described below. In one embodiment, at least some of data storage 310 might also be stored on another component of network computer 300, including, but not limited to, non-transitory media inside processor-readable removable storage device 336, processor-readable stationary storage device 334, or any other computer-readable storage device within network computer 300, or even external to network computer 300. Data storage 310 may include, for example, models 312 (e.g., heath score models or health sub-score), operations metrics 314, operations events 316, or the like.

Applications 320 may include computer executable instructions which, when executed by network computer 300, transmit, receive, or otherwise process messages (e.g., SMS, Multimedia Messaging Service (MMS), Instant Message (IM), email, or other messages), audio, video, and enable telecommunication with another user of another mobile computer. Other examples of application programs include calendars, search programs, email client applications, IM applications, SMS applications, Voice Over Internet Protocol (VOIP) applications, contact managers, task managers, transcoders, database programs, word processing programs, security applications, spreadsheet programs, games, search programs, and so forth. Applications 320 may include ingestion engine 322, modeling engine 324, resource management engine 325, analysis engine 326, other applications 327 that perform actions further described below. In at least one of the various embodiments, one or more of the applications may be implemented as modules or components of another application. Further, in at least one of the various embodiments, applications may be implemented as operating system extensions, modules, plugins, or the like.

Furthermore, in at least one of the various embodiments, ingestion engine 322, modeling engine 324, resource management engine 325, analysis engine 326, other applications 327, or the like, may be operative in a cloud-based computing environment. In at least one of the various embodiments, these applications, and others, that comprise the management platform may be executing within virtual machines or virtual servers that may be managed in a cloud-based based computing environment. In at least one of the various embodiments, in this context the applications may flow from one physical network computer within the cloud-based environment to another depending on performance and scaling considerations automatically managed by the cloud computing environment. Likewise, in at least one of the various embodiments, virtual machines or virtual servers dedicated to ingestion engine 322, modeling engine 324, resource management engine 325, analysis engine 326, other applications 327, may be provisioned and de-commissioned automatically.

In at least one of the various embodiments, applications, such as, ingestion engine 322, modeling engine 324, resource management engine 325, analysis engine 326, other applications 327, or the like, may be arranged to employ geo-location information to select one or more localization features, such as, time zones, languages, currencies, calendar formatting, or the like. Localization features may be used in user-interfaces and well as internal processes or databases. Further, in some embodiments, localization features may include information regarding culturally significant events or customs (e.g., local holidays, political events, or the like) In at least one of the various embodiments, geo-location information used for selecting localization information may be provided by GPS 340. Also, in some embodiments, geolocation information may include information providing using one or more geolocation protocol over the networks, such as, wireless network 108 or network 111.

Also, in at least one of the various embodiments, ingestion engine 322, modeling engine 324, resource management engine 325, analysis engine 326, other applications 327, or the like, may be located in virtual servers running in a cloud-based computing environment rather than being tied to one or more specific physical network computers.

Further, network computer 300 may also comprise hardware security module (HSM) 360 for providing additional tamper resistant safeguards for generating, storing or using security/cryptographic information such as, keys, digital certificates, passwords, passphrases, two-factor authentication information, or the like. In some embodiments, hardware security module may be employed to support one or more standard public key infrastructures (PKI), and may be employed to generate, manage, or store keys pairs, or the like. In some embodiments, HSM 360 may be a stand-alone network computer, in other cases, HSM 360 may be arranged as a hardware card that may be installed in a network computer.

Additionally, in one or more embodiments (not shown in the figures), network computer 300 may include an embedded logic hardware device instead of a CPU, such as, an Application Specific Integrated Circuit (ASIC), Field Programmable Gate Array (FPGA), Programmable Array Logic (PAL), or the like, or combination thereof. The embedded logic hardware device may directly execute its embedded logic to perform actions. Also, in one or more embodiments (not shown in the figures), the network computer may include a hardware microcontroller instead of a CPU. In at least one embodiment, the microcontroller may directly execute its own embedded logic to perform actions and access its own internal memory and its own external Input and Output Interfaces (e.g., hardware pins or wireless transceivers) to perform actions, such as System On a Chip (SOC), or the like.

### Illustrative Logical System Architecture

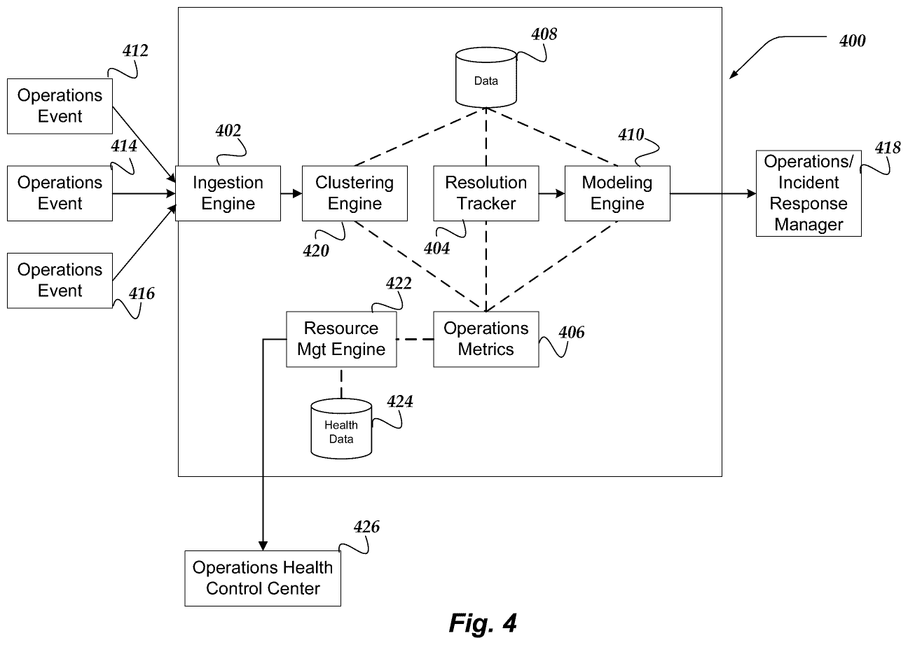

FIG. 4 illustrates a logical architecture of system 400 that provides operations health management in accordance with at least one of the various embodiments. In at least one of the various embodiments, a system for providing operations health management for entities or enterprises may comprise various components. In this example, system 400 includes, ingestion engine 402, resolution tracker 404, operations metrics 406, database 408, modeling engine 410, clustering engine 420, resource management engine 422, health data 424, operations health control center 426, or the like.

In one or more of the various embodiments, the operations health of a human responder and the health of a service as measured by the same human factors that cause the humans pain (or poor health) impacts performance. Specifically, unhealthy things like lack of sleep, inconsistent sleep cycles, alert fatigue, or the like, as measured by the data provided to ingestion engine 402 may cause poor cognitive performance (e.g. processing speed, focus, memory function, processing accuracy, or the like) as well as mental and psychological effects such as stress, anxiety, or the like.

In some embodiments, external conditions such as weather and other such environmental data collected via sensors (e.g., GPS 258 or GPS 340, or the like) may be represent in health models (which may account for factors such as weekly schedule, daily schedule, holidays, or the like) may be used as input sources for the computation of health specifically, but not necessarily performance.

In the context of IT Operation, performance could be measured as the time to resolve incidents, the number of incidents resolved, service downtime, business metrics, or the like, as discussed herein.

In at least one of the various embodiments, an ingestion engine such as ingestion engine 402 may be arranged to receive or obtain one or more different types of operations events provided by various sources, here represented by operations event 412, operations event 414, and operations event 416. In at least one of the various embodiments, operations events may be variously formatted messages that reflect the occurrence of events or incidents that have occurred in an organization's computing system. Such events may include alerts regarding system errors, warning, failure reports, customer service requests, status messages, or the like. operations events may be collected by one or more external services and provided to system 400. operations events, as described above may be comprised of SMS messages, HTTP requests/posts, API calls, log file entries, trouble tickets, emails, or the like. In at least one of the various embodiments, operations events may include associated information, such as, source, time stamps, status indicators, or the like, that may be tracked. Also, in some embodiments, operations events, may also be associated with one or more service teams the may be responsible for resolving the issues related to the operations events.

Accordingly, ingestion engine 402 may be arranged to receive the various operations events and perform various actions, including, filtering, reformatting, information extraction, data normalizing, or the like, or combination thereof, to enable the operations events to be stored and processed. In at least one of the various embodiments, operations events may be stored in database 408.

In at least one of the various embodiments, operations events may be provided by one or more organizations. In some embodiments, there may be several organization (e.g., 100's, 1000's, or the like) that provide operations events to the system. operations events from different organizations may be segregated from each other so that an organization may only interact with events that are owned by it. However, system 400 may be arranged to have visibility to all of the operations events enabling community wide analysis to be performed.

In at least one of the various embodiments, ingestion engine 402 may be arranged to normalize incoming events into a unified common event format. Accordingly, in some embodiments, ingestion engine 402 may be arranged to employ configuration information, including, rules, templates, maps, dictionaries, or the like, or combination thereof, to normalize the fields and values of incoming events to the common event format.

In at least one of the various embodiments, clustering engine 420, may be arranged to execute one or more clustering processes to provide one or more event clusters based on the normalized operations events. As described in more detail below, clustering engine 420 may be arranged to group operations events into event clusters based on one or more characteristics of the operations events.

In at least one of the various embodiments, resolution tracker 404 may be arranged to monitor the details regarding how the operations events are resolved. In some embodiments, this may include tracking the incident life-cycle metrics related to the operations events (e.g., creation time, acknowledgement time(s), or resolution time), the resources that are/were responsible for resolving the events, and so on. Likewise, operation metrics 406 may be arranged to record the metrics related to the resolution of the operations events. For example, operation metrics 406 may be arranged to compute various metrics, such as, mean-time-to-acknowledge (MTTA), mean-time-to-resolve (MTTR), incident count per resolvers, resolution escalations, uniqueness of events, auto-resolve rate, time-of-day of incidents, adjusting for multiple events per single incident, service dependencies, infrastructure topology, or the like, or combination thereof. Also, in at least one of the various embodiments, computed metrics may include time-to-discovery, time-to-acknowledgement, time-to-resolution, or transformations of these metrics, such as, mean, median, percentile, or the like. Further, one of ordinary skill in the art will appreciate that there are other relevant metrics that may be generated, measured, or collected. It is in the interest of clarity and brevity that the descriptions of additional metrics are omitted.

In at least one of the various embodiments, system 400 may include various user-interfaces or configuration information that enable organizations to establish how operations events should be resolved. (Not shown in FIG. 4) Accordingly, an organization may define, rules, conditions, priority levels, notification rules, escalation rules, or the like, or combination thereof, that may be associated with different types of operations events. For example, some operations events may be informational rather than associated with a critical failure. Accordingly, an organization may establish different rules or other handling mechanics for the different types of events. For example, in some embodiments, critical events may require immediate notification of a response user to resolve the underlying cause of the event. In other cases, the operations events may simply be recorded for future analysis.

In at least one of the various embodiments, modeling engine 410 may be arranged to use the various metrics associated with operations events, incidents, resolution of events, and so on, to produce one or more models that reflect the behavior of the operational system and organization. In at least one of the various embodiments, modeling engine 410 may be used to generate one or more operational models from one or more organizations that may be managed by system 400. Models for individual organizations may be provided as well as models for the community of organizations or sub-sections of the community.

Also, in one or more of the various embodiments, resource management engine 422 may be arranged to consume data from operations metrics 406 or database 408 to provide various metrics for measuring the health of operations personnel in an organization. Accordingly, in one or more of the various embodiments, resource management engine 422 may be arranged to provide various operations health scores that model the mental or physical health factors that may be associated with the persons that operate or maintain the monitored operations system for the one or more organizations monitored or measured by system 400. In one or more of the various embodiments, operations health scores may be provided based on an analysis of various measures of responder operational pain due to a number of issues that are known to impact emotional, physical, or mental health of the humans that actually response to incidents that may be associated with one or more operational events.

In one or more of the various embodiments, resource management engine 422 may be arranged to transform operations metrics or other information (e.g., operations events) into objects that may be analyzed to provide operations health scores. In some embodiments, operations health scores may be associated with different scopes or pivot that may be associated responders, team, or services. Each of responders, teams, or services may be individually evaluated by resource management engine 422 to provide one or more reports that indicate operations health. Operations health scores may be comprised of information that may measure the personal discomfort experienced by persons that actually have to respond to incidents. Briefly, in some embodiments, actions that may cause increased discomfort may include late night interruptions, dinner-time interruptions, weekend interruptions, or the like. Thus, in one or more of the various embodiments, responders that experience more of such action may have lower operations health scores than responders that have less of these type of uncomfortable interruptions.

Further, in one or more of the various embodiments, resource management engine 422 may be arranged to provide operations health scores that may be segmented in various dimensions such as, company size, market characteristics, number of employees, location, or the like, that enable the operations health of one or more organizations to be quickly and accurately compared.

Also, in one or more of the various embodiments, modeling engine 410 may be arranged to generate one or more health score models or health sub-score models based on one or more health metrics associated with one or more notification events that may be associated with the operations events. In one or more of the various embodiments, health score models or health sub-score models may enable resource management engines or analysis engines to generate health scores or health sub-scores. Also, in some embodiments, health score models or health sub-score models may enable the early prediction of adverse outcomes (e.g., employees quitting) that may be associated with low operations health.

In one or more of the various embodiments, operations health control center 426 may be arranged to provide user-interfaces that provide interactive reports that enable organizations to view operations health scores. In some embodiments, one or more of the interactive reports provided by operations health control center 426 may enable users to explore the relationships between their operations and individual operations health sub-scores that contribute to an overall organization level operations health score.

Furthermore, in at least one of the various embodiments, since client computer 200 or network computer 300 is arranged to include one or more sensors for determining geolocation information (e.g., GPS), monitoring electrical power conditions (e.g., voltage sensors, current sensors, frequency sensors, and so on), monitoring weather (e.g., thermostats, barometers, anemometers, humidity detectors, precipitation scales, or the like), or the like.

For example, in at least one embodiment, geolocation information (such as latitude and longitude coordinates, or the like) is collected by a hardware GPS sensor and subsequently employed in the computing of performance metrics, operations models, or the like. Similarly, in at least one embodiment, weather information (such as temperature, atmospheric pressure, wind speed, humidity, or the like) is collected by a hardware weather sensor and subsequently employed in the computing of performance metrics, operations models, or the like. Additionally, in at least one embodiment, electrical power information (such as voltage, current, frequency, or the like) is collected by a hardware electrical power sensor and subsequently employed in the computing of performance metrics, operations models, or the like. Also. operations events may be modified to include geolocation or sensor information. Accordingly, performance metrics and operations models may be categorized or compared across different conditions or locations. For example, hot and cold weather extremes may impact the values of one or more metrics or models. Likewise, in at least one of the various embodiments, system 400 may be arranged to determine one or more localization features based on the geolocation information collected from its GPS systems, sensors, network, network interface, or the like, or combination thereof.

Also, in at least one of the various embodiments, sensing geolocation information provided by one or more geolocation devices may be employed to perform one or more actions, such as: providing a modification of the one or more metrics or models based at least on the sensed information; or localizing the one or more recommendations based at least on the sensed information. For example, geolocation information may be used to account for local holidays, or the like, that result in interrupt events causing more pain for responders in different geographic locations. In some embodiments, some or all geolocation information may be provided or supplemented based on user input, configuration information, policy rules, or the like.

In one or more of the various embodiments, innovations described herein decrease operational pain and increase system efficiency through monitoring the health of personnel in organizations. In some embodiments, this may be accomplished through measuring, monitoring, reducing meaningful incident behavior, or the like, or combination thereof, across an organization and using it to inform necessary changes in the organizational operations to reduce operational pain points.

Because poor on-call experiences often lead to sleep deprivation, heightened levels of frustration, an inability to work effectively, or the like. Accordingly, if not addressed in a timely manner, these pain points may create operational inefficiencies, and cause top talent to leave an organization in search of a healthier work environment-both of which can severely impact an organization. For example, with the cost of replacing skilled IT professionals reaching $300,000 each, it is critical that organizations find ways to prioritize efforts to maintain a healthy working environment.

Further, in one or more of the various embodiments, one or more operational health metrics may be linked directly to the affected responders, teams, or services. Also, in one or more of the various embodiments, the events that may be associated with the one or more operational health metrics may be linked or associated with the operations events they are derived from.

In one or more of the various embodiments, a resource management engine may be arranged to perform actions, including, incident response efficiency self-assessments. Accordingly, in one or more of the various embodiments, an organization may be enabled to measure their baseline incident notification and response behavior and draw meaningful conclusions based on historic trends. In one or more of the various embodiments, such information may provide value to an organization as it may help the organization gain insight into one or more operations metrics, such as, operational efficiency, alert fatigue, work allocation across teams and responders, or the like. For example, in one or more of the various embodiments, each team's notification time of day distribution may be compared against what may be expected with respect to its specific workload, function, service ownership, schedules, or the like.

Accordingly, for example, in some embodiments, an organization's leadership may want to flag anomalous responders, teams, services, or the like, with a mean notification time of day which may be incongruent with specific workloads or functions of the team or individual. For example, in some embodiments, an organization may query why the notification windows for a particular responder, team, or service comes at certain times, and how that may adversely affect incident response time, or over time, increase likelihood of employees (e.g., affected responders) to quit their job. In one or more of the various embodiments, insights like this may be made across many other dimensions using other measurements or metrics (as discussed in more detail below).

In one or more of the various embodiments, a resource management engine may be arranged to perform actions, such as, incident response efficiency monitoring, or the like. Accordingly, in some embodiments, an organization may employ the resource management engine to analyze its daily/weekly incident notification and response behaviors by comparing them to historical distributions in time. in one or more of the various embodiments, such analysis may enable the organization to evaluate if it is “healthy” this week, or is it more or less healthy than last week/month/year, or the like. Further, in one or more of the various embodiments, resource management engines may be arranged to perform monitoring that may be used to track statistical anomalies or distribution shifts that may indicate inefficiencies that need immediate attention and process improvement.

In one or more of the various embodiments, a resource management engine may be arranged to perform actions, including industry benchmarking. In one or more of the various embodiments, industry benchmarking may enable an organization to compare its own operational health trends against other organizations of similar type, size, revenue, and industry segment. In some embodiments, this type of information could show where an organization's IT operations may be deficient, unevenly loaded, or fatigued thereby impairing performance, efficiency, productivity, profitability, or the like.

In one or more of the various embodiments, a resource management engine may be arranged to perform actions to actively manage the health of its IT operations. In one or more of the various embodiments, ergonomic data or metrics collected by a resource management engine may be used to intelligently inform proper system management decisions to increase human well-being across the an organization's workforce to optimize overall system performance. In one or more of the various embodiments, “system” may be defined as the coupled interaction between the IT Operations infrastructure and the people who build, operate, or maintain it. In one or more of the various embodiments, health management may include using the information collected or provided by a resource management engine to recognize areas in the organization that need improvement or repair. In some embodiments, resource management engines may be arranged to automatically make one or more needed repairs. For example, in some embodiments, resource management engines may be arranged to automatically modify work schedules, maintenance schedules, or the like, in response to discovered issues. Additional examples of informed updates based on resource management engines metrics or reports include reconfiguring alert/notification settings, modifying alert rules, using noise suppression mechanisms, re-routing alerts, reallocating workloads for responders or teams for even balance across the organization, or the like, or combination thereof. In one or more of the various embodiments, resource management engines may be arranged to execute a continuous feedback loop that may provide continuous overall system optimization.

In one or more of the various embodiments, resource management engines may be arranged to describe operations health in various contexts, including: services, teams, responders. In some embodiments, for services, resource management engines may be arranged to aggregate of the overall health as reported on Insights Service Graphs for defined time periods (e.g., interactive reports). For teams, resource management engines may be arranged to aggregate of the overall health as reported on Insights Team Graphs for defined time periods (e.g., interactive reports). And, for responders, resource management engines may be arranged to aggregate of the overall health as reported on Insights Responders Graphs for defined time periods (e.g., interactive reports).

In one or more of the various embodiments, resource management engine may be arranged to compile information from a variety of sources to employ for providing operations health scores. In one or more of the various embodiments, operational health may be analyzed across various parameters, including, sleep interruptions, dinner interruptions, successive days of notifications, push vs. email notifications, weekday vs weekends, or the like, or combination thereof.

In one or more of the various embodiments, operations health scores may be considered statistical representations that model how and when responders are notified across a statistical distribution providing a holistic view of their incident response trends. Accordingly, in some embodiments, if operations health scores are aggregated, they may provide organizations a profound understanding of what is happening across their teams and services.

In one or more of the various embodiments, resource management engines may be arranged to provide operations health scores that model operational health based on a variety of statistical features, including: measure of mean hour of day notifications are received; proportion of interrupting (i.e. non email) notifications during sleep hours; proportion of interrupt (i.e. non email) notifications during dinner hours; notification variation throughout the day (in hours); proportion of email notifications; proportion of interrupt (i.e. non email) notifications during weekends; proportion of days across time period with non-email notifications during sleep hours; proportion of days across time period with non-email notifications during dinner hours; proportion of days across time period with non-email notifications during evening hours; proportion of days across time period with non-email notifications during weekends; measure of successive days across time period with non-email notifications during sleep hours; measure of successive days across time period with non-email notifications during dinner hours; measure of successive days across time period with non-email notifications during evening hours; measure of successive days across time period with non-email notifications during weekend; measure of notification count with respect to distribution of notification counts across the organization; or the like, or combination thereof.

In one or more of the various embodiments, resource management engines may be arranged to collect parameters or metrics that may be mathematically combined to provide operations health models. In one or more of the various embodiments, the weights (e.g., coefficients) associated with the various parameters may be adjusted based on configuration information. In some embodiments, resource management engine may include a plurality of models, with different models directed or tailored to different industries, markets, company size, organization composition, or the like. Likewise, in one or more of the various embodiments, resource management engines may be arranged to enable organizations to employ or derive models that are customized to their needs. In some embodiments, operations health scores may be normalized to range from 0 to 100 and may be computed for each responder, team, and service for an organization.

In one or more of the various embodiments, resource management engines may be arranged to employ feedback from machine-learning or classification systems, or the like, that correlate operational outcomes. such as acknowledge time, resolve time, churn time, or the like, with operations health parameters. Accordingly, in some embodiments, resource management engines may be arranged to dynamically modify operations health score models in real-time to automatically adapt one or more operations health score models to changes discovered by the machine-learning or classification systems.

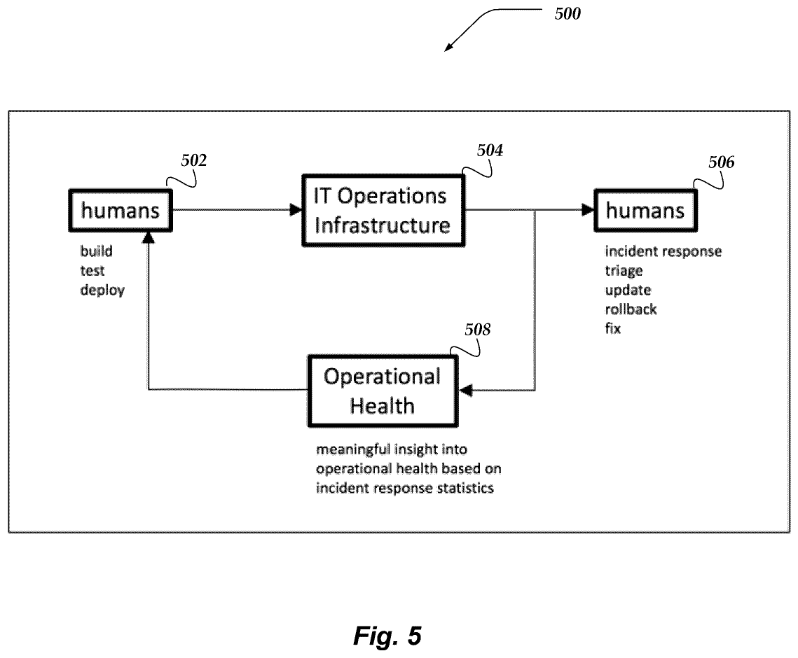

FIG. 5 illustrates a logical architecture of system 500 that provides operations health management in accordance with at least one of the various embodiments. System 500 provides a high level overview of how the operational health of an organization may be related to how it manages IT operations. In some embodiments, humans 502 may build, test, or deploy systems or applications comprising IT operations infrastructure 504, humans 504 may be the responsible resources that provide incident response, triage, updates, rollbacks, fixes, or the like, that may be associated with maintaining IT operation infrastructure 504. Accordingly, in one or more of the various embodiments, operational health 508 represents one or more operational health models, such as, operations health scores, that may provide an organization meaningful insight into its operational health based on operations metrics, such as, incident response statistics, or the like.

In one or more of the various embodiments, resource management engines may be arranged to relate meaningful data from interactions between responders activities and IT infrastructure operations to create an optimization cycle to enhance efficiency.

In one or more of the various embodiments, resource management engines may be arranged to measure and respond to the causes-and-effects associated with responder interaction with an organizations IT operations. In one or more of the various embodiments, responders may interact with the infrastructure in two or more ways, including by making changes to it, responding to the infrastructure when it degrades or fails, or the like.

In one or more of the various embodiments, the way in which the responders interact with the infrastructure when it degrades or fails may be captured by incident notifications (e.g. operation events) and response data, which may be reduced in a meaningful statistical manner to represent inefficiencies in the organization's IT operations. In one or more of the various embodiments, these incident notification and response statistics may be reflected in an operations health score, which holistically describes the amount of pain the responders, teams, or services of an organization may be experiencing. Thus, in some embodiments, this may influence the business of an organization by impacting the organization's ability to maximize its revenue or take full advantage of an optimized organization and system with its inherent coupling between its infrastructure and its people.

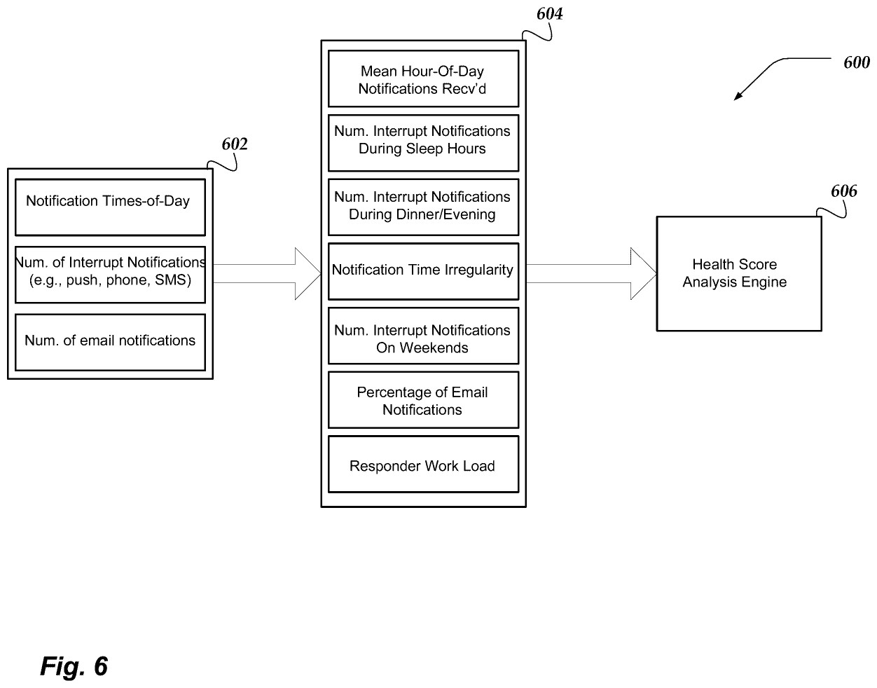

FIG. 6 illustrates a functional architecture of system 600 for providing model inputs to a scoring engine in accordance with one or more the various embodiments. In one or more of the various embodiments, metrics 602 associated with notification events may be characterized to provide one or more health sub-scores 604. Accordingly, in one or more of the various embodiments, health sub-scores 604 may be provided to an analysis engine, such as, analysis engine 606 the may be arranged to provide a health score based on health sub-scores 604.

In one or more of the various embodiments, operations health scores may be provided based on a feed-forward multi-dimensional parametric algorithm that accepts the one or more health sub-scores. In one or more of the various embodiments, operations health scores are designed to be a measure of responder operational pain. In some embodiments, the analysis engine may be arranged to account for one or more responder health issues. Also, in some embodiments, analysis engines may be arranged to distinguish health related events that occur over different periods of time. Accordingly, in some embodiments, responder health scores may be arranged to flexibly represent each specific on-call experience. Note, the method disclosed herein includes a descriptive summary statistical approach. However, one of ordinary skill in the art will appreciate that these innovations contemplate other possible and viable methods to compute health, such as inferential statistical modeling, maximum likelihood estimation, Fisher scoring, or the like.

In one or more of the various embodiments, some non-limiting examples of pain points may include:
Frequent notifications which are distractions that occur at inopportune times, i.e. sleep hours, dinner time, or weekends;
Alert volume which inundates a responder, and is anomalous with respect to all other responders across the organization;
Notification variation throughout a typical day as a measure of how irregular and spread out notifications are across the day;
Proportion of days across a period with interrupt notifications;
Successive days with notifications during off-work hours or weekends;
Operational inefficiencies like frequent system failures, misconfigurations, and user account notification settings;
Low maturity indicators such as the percentage of emails which are notifications;
Number of re-assignment counts;
Number of timeout counts;
Holiday notification trends; or the like.

In one or more of the various embodiments, resource management engine may be arranged to characterize some notification events as interrupt events. Interrupt events may be associated with notifications triggered by events or alerts that are configured to alert responders and shift the focus of those responsible responders away from whatever they are doing. In other words, interrupt events are notification events that may be or appear to be related to critical problems which must be addressed immediately, no matter if the responder is sleeping, or it's a weekend day at the park with their kids, or at a holiday party with friends. Accordingly, for example, interrupt events may include SMS, phone calls, pager pages, push notifications, or the like. In comparison, an example of non-interrupt notifications include email messages or other notifications provided for information purposes rather than requiring immediate response from a responder.

In one or more of the various embodiments, an operations health score model may be arranged to consider these notification statistics (e.g., health sub-scores) which may have varying on-call implications on the responder. The operations health score model may operate to transform each health sub-score a normalized parameter. In some embodiments, each health sub-score may be weighted according to its relevance as it relates to human pain. The combination of the normalized or weighted health sub-scores may provide a single operations health score for a given responder, team, department, or organization.

In some embodiments, human pain may either directly or indirectly be measured by a function of human performance indicators. In the context of IT Operations, an example of human performance can mean average time to resolve incidents.

In other contexts where a person receives interrupt notifications on a regular basis for their job (other use cases of modern incident management in other fields such as hospitals, emergency response, power and energy systems management, or the like), human pain could be measured in terms of ability to do normal daily functions, or meet other job performance metrics, or employee attrition rate.

In other contexts where a person receives interrupt notifications on a regular basis as a normal human being on their personal device, human pain could be measured as a rise in stress levels, blood pressure, anxiety, or the like.

For example, to determine a particular responder's operations health or an operations health score for a particular service across a given period of time (e.g. day/week/month/year) notification event data for the responder or service may be captured and reduced to statistical features that may be combined to produce an operations health score.

In one or more of the various embodiments, a resource management engine or analysis engine may be arranged to provide an operations health score model that provides an operations health score (H) as shown below:

$$H = 100 \times ( 1 - \frac{1}{N - 1} {\sum }_{i = 1}^{N} {w}_{i} {x}_{i} ) - {w}_{N} {x}_{N}$$

In one or more of the various embodiments, the relevance of each health sub-score may be determined by means of a second model that approximates the relationship between the health sub-score and various human factors, for example, consider:

$$Y = f ( X ) \\ X = \{ \begin{matrix}{x}_{1} \\ {x}_{2} \\ ⋮ \\ {x}_{N}\end{matrix} \}$$

where:
Y=the human factor response target being modeled, e.g. employment tenure length (in months) of responders;
X=input vector of notification parameters derived from the raw notification data; and
f(X) may be a model that approximates the relationship between X and Y, which includes information about how each input of X relates to Y.

Examples of model f may include parametric and structured models such as linear regression, nonlinear regression, logistic regression, or the like, or combination thereof. In some embodiments, operations health models or operations health sub-score models may also include other forms of models that fit to numerically varying response targets, such as artificial neural networks, support vector transforms, or the like.

In one or more of the various embodiments, if the human factor can be classified into categories, the model may include Bayesian classifications, nearest neighbors, discriminant analysis, or the like. In any case, internal model parameters such as weighting coefficients or classification boundaries can be leveraged for insertion into the health score model since the health sub-scores provided by operations health sub-score models, such as, f, may be proportional to the relevance of each notification parameter. Accordingly, in some embodiments, the health score may be used to provide health score timelines and benchmarks that may be correlated with one or more tangible human factors.

In some embodiments, human factors may be collected and used in various ways, for example:

1. Employee tenure length. A human factor model that is fit to employee tenure length means that may provide organizations with knowledge of when an employee has an elevated risk of leaving the organization permanently due to human pain. In one or more of the various embodiments, the broader the time scale (e.g. month or quarter), the more accurate the estimate.

2. Performance measures such as incident acknowledge and resolve time, or other productivity measures. Like employee tenure, in some embodiments, a human factor model that approximates the relationship between operations health sub-scores and productivity enables predictions related to if a responders poor on-call experience translates to the reduced ability to be productive on the job.

3. Medical and psychological factors such as fatigue, mental anxiety, high blood pressure, stress, PTSD, or the like. In some embodiments, model may take advantage of existing studies from the medical, psychological, social science communities. Accordingly, by approximating operations health scores with known medical and psychological factors, this model may be used to ensure that responders' well-being is properly assessed and managed.

4. Direct feedback from responders such as employee survey results that measure on-call pain may also be used as a response variable for the human factors model.

In one or more of the various embodiments, resource management engines may be arranged to employ incident response data that may be associated with one or more organizations to numerically represent each organization's operational health score in various contexts, such as, responders, teams composed of responders, services owned or operated by responders, overall organization health, or the like.

In one or more of the various embodiments, resource management engines may be arranged to provide operations health scores by executing actions to produce a holistic measure of responder operational pain that may be based on a number of different operations issues. Some examples of pain points may include: frequent notifications which are distractions that occur at inopportune time (e.g., sleep hours, dinner time, weekends, or the like); alert volume (or rate) which may inundate responders, and may be anomalous with respect to other responders across the organization; notification variation throughout a typical day; operational inefficiencies including frequent system failures, misconfigurations, user account notification settings, or the like; number of re-assignment counts; number of timeout counts; holiday notification trends; or the like.

In one or more of the various embodiments, resource management engines may be arranged to provide operations health scores based these various metrics. In some embodiments, resource management engines may be arranged to perform actions, including transforming the one or more metrics into normalized parameters. In some embodiments, each parameter may be weighted according to their relevance as it relates to human pain and combined to provide a single operations health score.

In one or more of the various embodiments, the relevance of each parameter may be determined using one or more statistical models, that may approximate the relationships between the operations health score parameters and human pain. In some embodiments, these may include churn time on the job as well as slow incident acknowledge and resolve time. In one or more of the various embodiments, resource management engines may be arranged to employ one or more statistical models, based on regression, Bayesian classification, discriminant analysis, or the like. In some embodiments, coefficients employed in the one or more statistical models may be proportional to the relevance of each parameter.

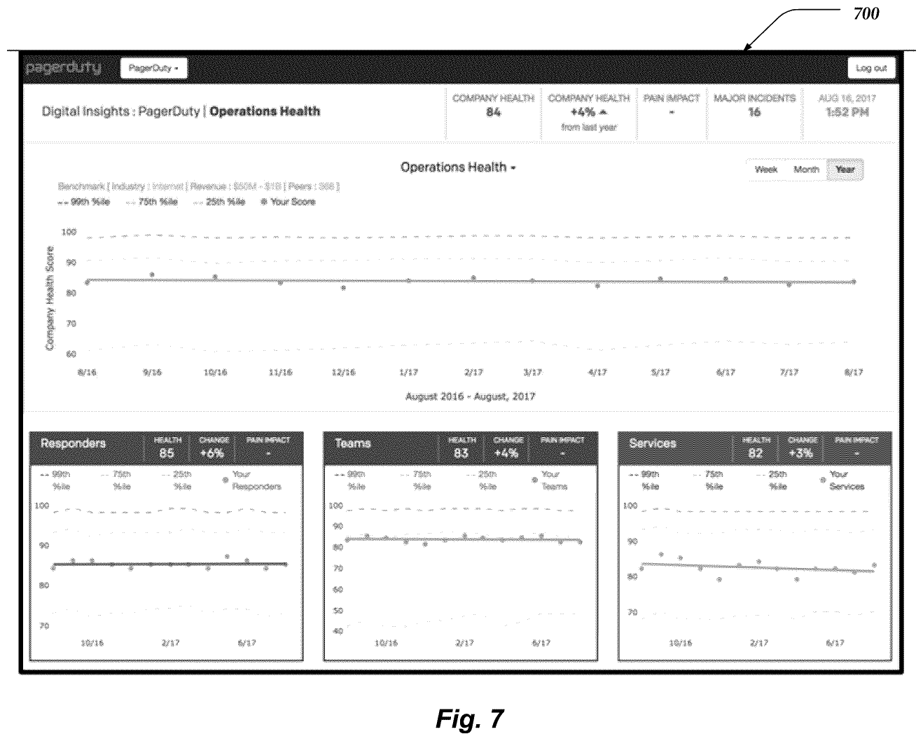

FIG. 7 illustrates a logical representation of interactive report 700 that enables organizations to manage their operations health in accordance with at least one of the various embodiments. In one or more of the various embodiments, resource management engines may be arranged to provide various interactive reports that enable people in an organization to view various aspects its operational health. One of ordinary skill in the art will appreciate that interactive reports may be provided using various arrangements, visualization, or formats. Also, one of ordinary skill in the art will appreciate that additional visualizations using or representing a variety of health information, operations information, monitored metrics, or the like, may be included in interactive reports provided by resource management engines. Accordingly, for brevity and clarity, interactive reports, such as, interactive report 700 are presented herein as non-limiting illustrative examples to represent the plurality of interactive reports that may be provided by resource management engines.

In some embodiments, the operations health scores are displayed in responder, team, and service contexts. In one or more of the various embodiments, operations health scores may be provided for each responder (individual), each team of responders, and finally for each service that causes responder pain. For each of these pivots, the resource management engine may be arranged to use the same notification event statistics to compute health scores that represent the human impact of the on-call experience.

In addition, in one or more of the various embodiments, patterns of operations health may be viewed across different timelines, such as, days, weeks, months, quarters, years, or the like. In one or more of the various embodiments, the longer the time period, the more statistically representative the health score. Also, in some embodiments, linear trends may be tracked across each timeline, and the user can view the percentage change in health scores from the previous time period.

Also, in some embodiments, benchmark data may be provided to illustrate how the health of any responder, team, service, or overall organization compares to industry peers as defined by business segment, revenue, or the like.

In one or more of the various embodiments, resource management engines may be arranged to display or represent operations health scores timelines or benchmarks contexts associated with responders, teams, or services. In one or more of the various embodiments, system 900, described below, illustrates how a resource management engine may be arranged to enable an organization to interact with health information provided by a resource management engine.

Further, in this example, (interactive report 700), the top plot represents the overall organization's operations health score over time. These time varying plots can be viewed in different time periods or time buckets (e.g., week, month, year, or the like). Also, in some embodiments, along with each time series plot benchmark, additional information may be displayed enabling organizations to compare their operations health scores with their peer organizations, as determined by company size, industry segment, or the like. In this example, interactive report 700 includes the three visualizations that plot each of the different contexts: overall responder scores, overall teams scores, and overall services scores.

Accordingly, in one or more of the various embodiments, resource management engines may be arranged to enable users to drill down through the displayed visualizations to gain further insight into which teams may be lowering the organization's overall score, or which responders may be most unhealthy, or the like.

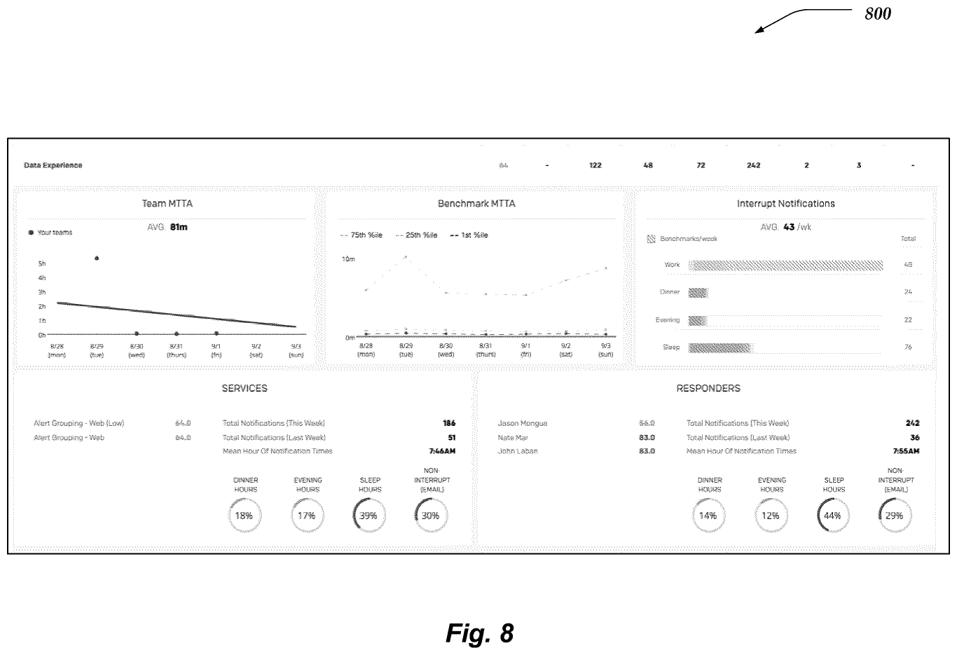

FIG. 8 illustrates a logical representation of interactive report 800 that enables organizations to manage their operations health in accordance with at least one of the various embodiments. Interactive report 800 represents an embodiments of an interactive report that shows operations health score trends for an organization. As described above for report 700, resource management engines may be arranged to provide interactive reports comprised of various visualizations. In this example, interactive report 800 includes a visualization that show operations health score trend lines for various contexts as well as relative changes, that reflect increases or decreases in an organization's operations health scores.

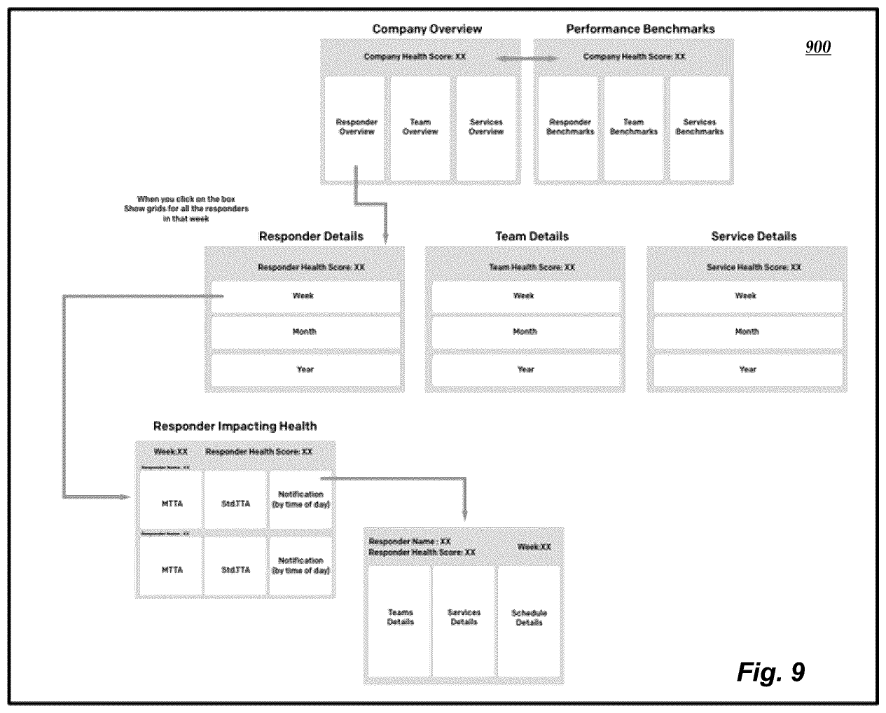

FIG. 9 illustrates a logical schema of system 900 that includes data structure for managing operations health in accordance with one or more of the various embodiments. In one or more of the various embodiments, system 900 shows high level data relationships between some of the entities provided by a resource management engine that may enable operations health scores of one or more organizations to be viewed or analyzed by an organization. Accordingly, in one or more of the various embodiments, resource management engines may be arranged to provide interactive reports that provide linked or related visualizations that a user may drill down (or across) to view different contexts or aspects associated their organization's operations health score.

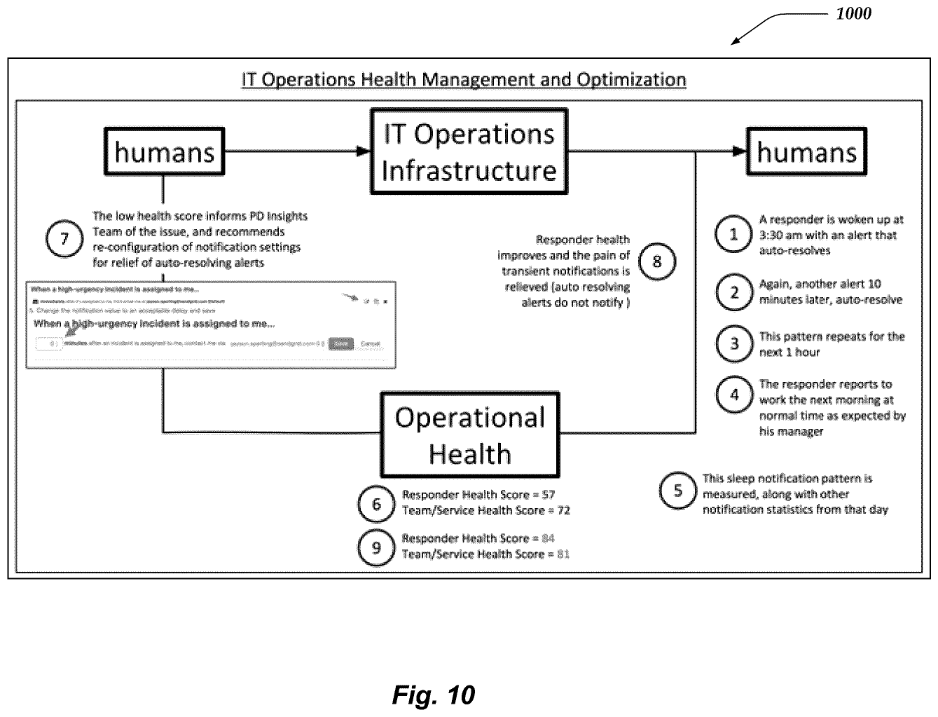

FIG. 10 illustrates an overview of process 1000 that shows an example of how IT operations in an organization may impact an organization's operations health scores in accordance with one or more of the various embodiments. For brevity and clarity, process 1000 illustrates a real-life use case of the measurement, diagnostics, and prognostics to improve the health of a responder. This example shows the immediate value that monitoring responder health may have on both the human well-being of the responder as well as for the organization. This example represents the common case of an IT operations responder forced to absorb a large amount of non-actionable notifications on a daily basis. In this non-limiting example, the operational pain is caused by these notifications being triggered by alerts which auto-resolve themselves in a matter of minutes. At step 1, in this example, a notification is triggered at 3:30 am based on an alert from a management system monitoring the infrastructure.

In response to this, the responder is woken by the notification on their phone and logs into his machine to assess the issue. Once the responder logs in, they may be enabled to see that the alert was auto-resolved by the monitoring integration. Annoyed, the responder shuts his laptop and goes back to bed. At step 2 of this example, just as the responder gets back to sleep, his phone notifies him again with a new alert for the same or similar issue. At step 3 of this example, these notifications continue throughout the rest of the night, leaving the responder exhausted, irritable and annoyed the following day. At step 4 of this example, the responder reports to work as expected on-time and is responsible to take care of his normal duties. At step 8 of this example, this sleep time notification pattern is measured, along with other notification statistics from that day's work. At step 6 of this example, as a result of the sleep interruption pattern, as well as that day's notification trends, the responder's operations health score is calculated to be a paltry 57. Accordingly, this poor responder health value also brings down the health of his team and the services he supports to a 72.

In one or more of the various embodiments, resource management engines may be arranged to model this type of pathology using the operations health score model and may be used as a diagnostic indicator of this poor operational health for responders, services, or teams. Accordingly, in some embodiments, the operations health scores and its associated underlying operations metrics may be used to improve the monitoring functions of the organization by identifying or suggesting ways to intelligently improve how the system is monitored. For example, in some embodiments, operations metrics associated with operations health scores may indicate changes such as buffering notifications prior to sending should be implemented. For example, if a notification is buffered for five minutes before sending, these type of recurring non-actionable alerts may be stopped short of becoming notifications thereby increasing the responder health to 84 (for example), and his team's health to 81.

In one or more of the various embodiments, resource management engines may be arranged to enable organizations to implement small, incremental fixes to increase responder health in the short-term. With fixes such as these made over time and across an organization, overall operational efficiency may be improved. Accordingly responders and teams may be more in sync with the system they are operating and managing, and their respective services may be healthier overall since operators may be more available to spend time improving them rather than being distracted by unnecessary interruptions.

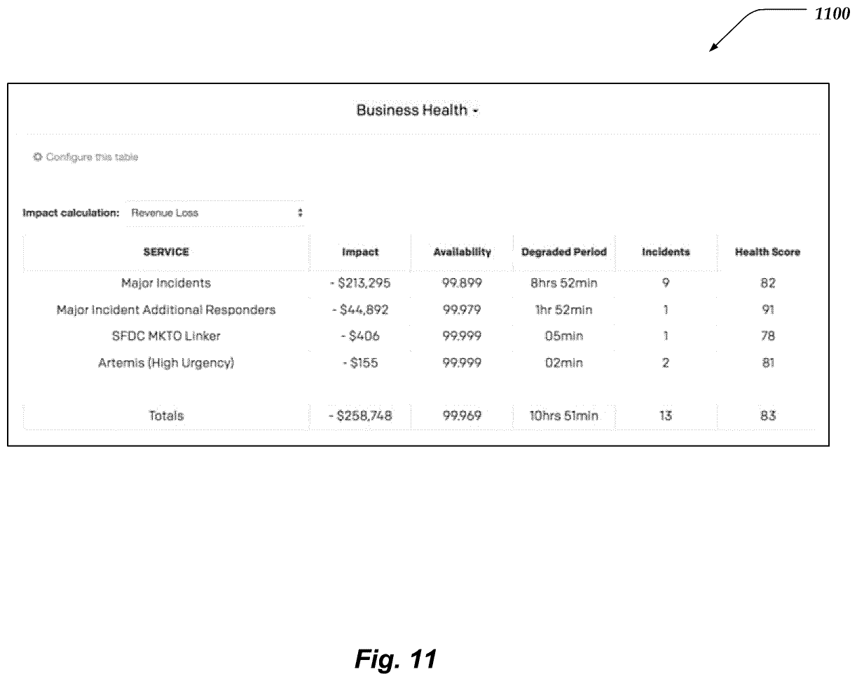

FIG. 11 illustrates a logical representation of interactive report 1100 that shows how an organization's operations health scores may impact business health of an organization in accordance with one or more of the various embodiments. In one or more of the various embodiments, resource management engines may be arranged to provide information measuring how operational health information may be used to measure impact to the business. In one or more of the various embodiments, resource management engines may be arranged to provide business health insights that may be derived from a combination of both operational and business metrics. In one or more of the various embodiments, these metrics may include: the overall operations health score for a critical service or feature; revenue lost by that critical service; the service availability, in terms of reliability; the duration of degraded performance of that service or feature; the number of major incidents having to do with the service or feature; or the like.

In one or more of the various embodiments, by using this information together, an organization may be enabled see what incidents have the largest impact to its business as a way of prioritizing which services and teams receive focus, attention, repair, optimization, or the like. Accordingly, in one or more of the various embodiments, resource management engines may be arranged to provide one or more interactive reports that may provide links between critical services, teams or responders associated with the service, the associated critical metrics/scores for those elements, or the like.

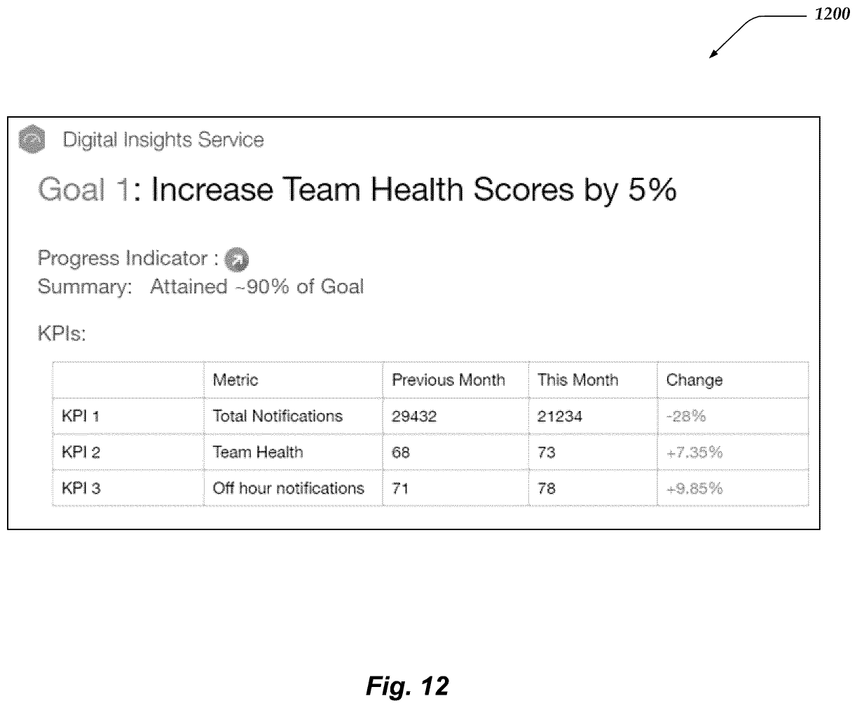

FIG. 12 illustrates a logical representation of interactive report 1200 that shows how a portion of an organization's operations health score(s) key performance indicators (KPIs) have changed over a given time period in accordance with one or more of the various embodiments. Accordingly, as mentioned above, in one or more of the various embodiments, resource management engines may be arranged to provide various interactive report, such as interactive report 1200. This example, represents a report that compares month-to-month changes for the KPIs. In this example, the KPIs are generically labeled, but in some embodiments, the labels may be arranged to reference that particular indicators being reported on.

In one or more of the various embodiments, resource management engines may be arranged to enable users or organizations to define one or more goals and to associate or define one or more KPIs with the one or more goals. Accordingly, in some embodiments, the one or more KPIs may be used to measure or illustrate progress towards the one or more goals.

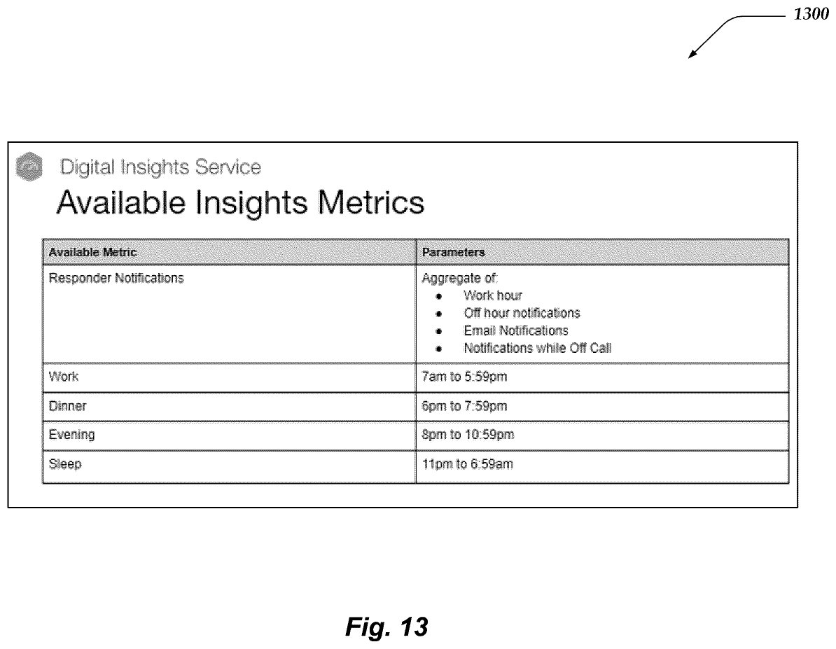

FIG. 13 illustrates a logical representation of interactive report 1300 that includes a few example metrics and parameters that may provide various insights into an organization's operational health in accordance with one or more of the various embodiments.

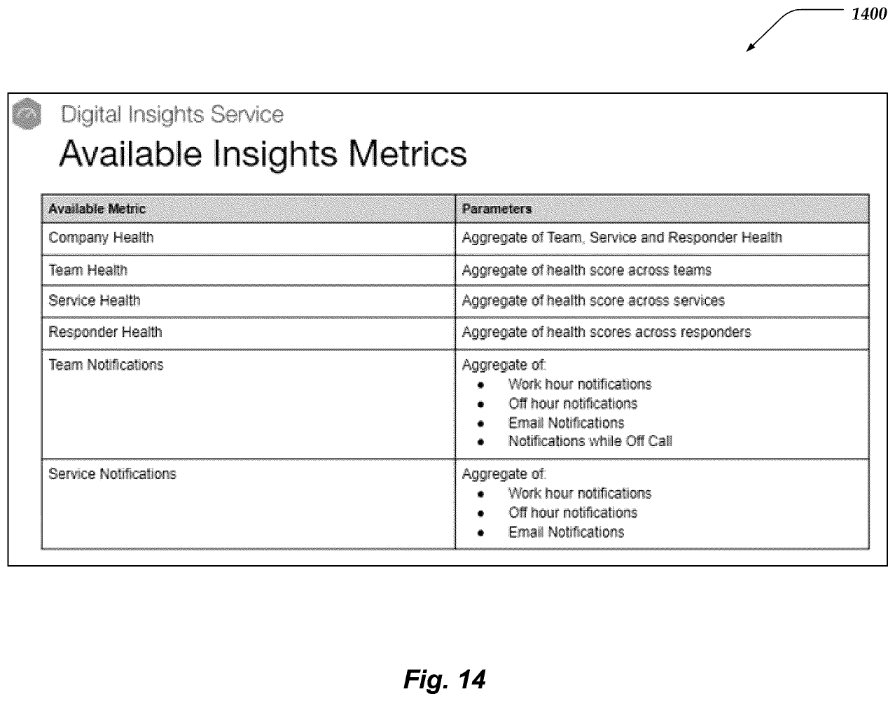

FIG. 14 illustrates a logical representation of interactive report 1400 that shows how additional insights into an organization's operations may be related to an organization's operations health scores in accordance with one or more of the various embodiments.

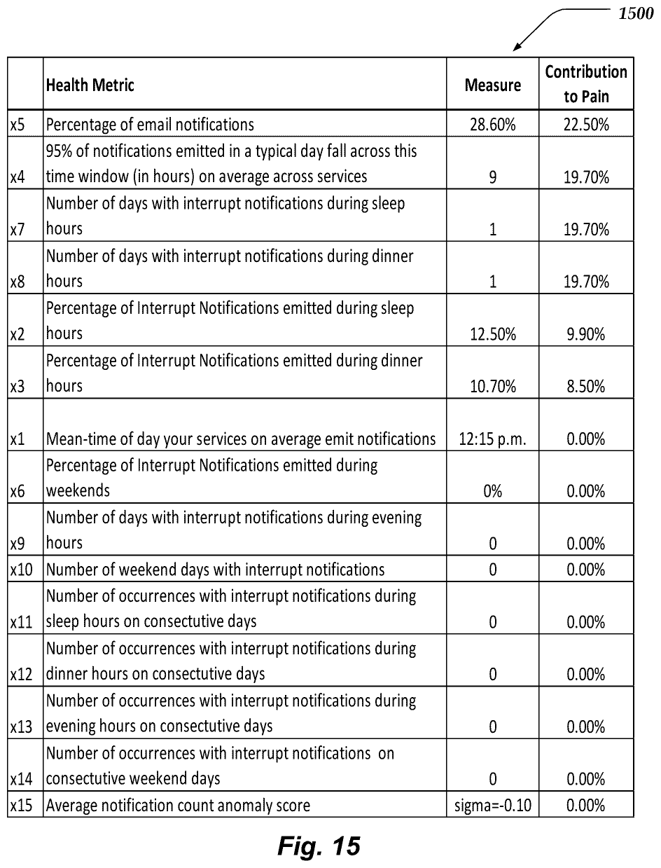

FIG. 15 illustrates a logical representation of interactive report 1500 that shows contributing sources of pain for individual responders and services in accordance with one or more of the various embodiments. In one or more of the various embodiments, resource management engine may be arranged to enable users to inspect the breakdown of contributing sources of pain as they relate to an operations health score. For example, interactive reports provided by a resource management engine may be arranged to answer questions, such as, “where, why, and how poor is the health of an organization? What are the contributing health factors as an organization? Which responders/services are have the lowest scores across the organization? Looking at a particular responder/service over this given period of time, what is the breakdown of contributing health sub-scores for a specific responder's/service's health?”

In one or more of the various embodiments, answering these questions may enable organizations to improve their understanding of the meaning behind the operations health score for a responder or service. This information may provide a next level of insight that enables the organization to develop a plan for improving responder health.

Accordingly, in one or more of the various embodiments, these interactive reports may help leaders understand what aspects of their operations cause their responders the most pain and why. Also, the detailed reports may enable organizations to discover how or which services may be contributing to the operational pain experienced by responders. The operations health score also allows organizations to view the contributing sources of pain for individual responders and services.

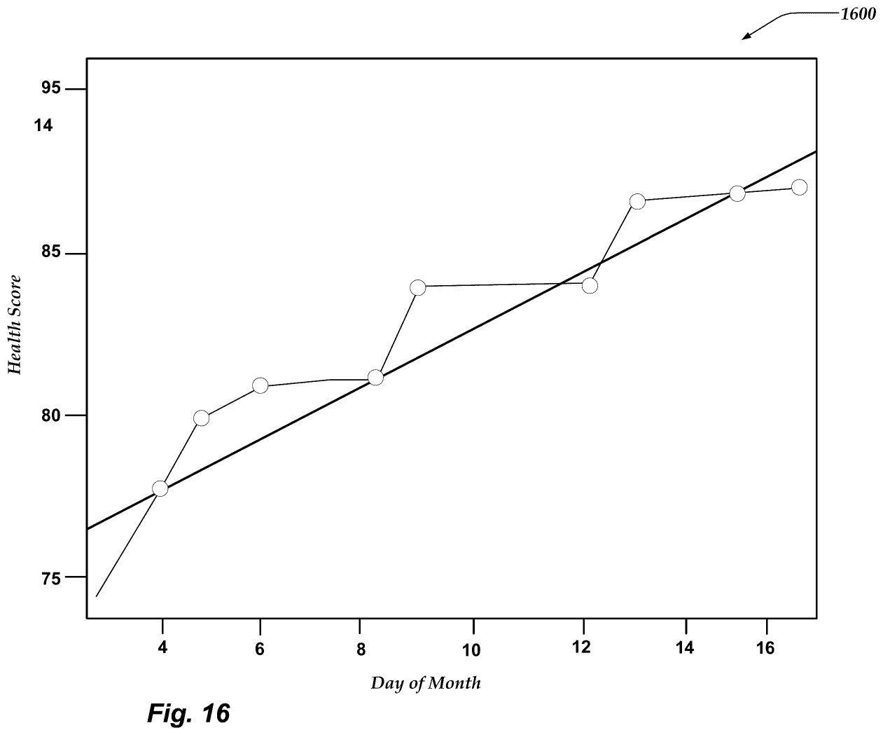

FIG. 16 illustrates a logical representation of interactive report 1600 that shows health score with the remediation trends related to contributing sources of pain for individual responders or services in accordance with one or more of the various embodiments.

In one or more of the various embodiments, resource management engines may be arranged to provide actionable data-centric information about a company's employees and their digital infrastructure. Accordingly, in some embodiments, resource management engines may be arranged to deliver quantified health information pertaining to an organization's responders, teams, services, or the like. In some embodiment, this may provide these organizations the power to make informed and intelligent business decisions to improve and maintain competitiveness.

In one or more of the various embodiments, resource management engine may be arranged to provide operations health data that enables organization leaders to make informed decisions by analyzing trade-offs, for example:

1. The manager or leader of a team may look at the health patterns over time to see waves of low health due to heavy load cycles, and periods of recovery when the system is working without performance degradation. The manager would use this knowledge of his team's on-call notification pain and relief cycles to gage how to best schedule rotations to keep his team productive and well-rested because the focus is on keeping the job sustainable. Automated intelligent scheduling of teams can also be realized in how it may be used to measure by using the operations health data as feedback to inform the appropriate on-call schedules.

2. If in the event that on-call cycles are not presenting enough time to recover and regain improved health for a responder, then the manager or team lead can use this knowledge to schedule an off-call period for the required duration in order to improve that responder's health. This is called off-call remediation, and is the next best action to take when an employee is inundated by the on-call experience because the rate of remediation is higher than on-call relief periods. In other words, when off-call a responder can focus all of his/her efforts on the primary job responsibilities while at work, and resume a normal life during weekends and off-hours.

For example in one representative scenario, where an on-call responder was inundated by alerts due to a down service during the first 3 days of the month:
The huge volume of notifications received by this responder measured as an anomaly with respect to the distribution of notifications received by his co-workers across his organization
Notifications received throughout the night were interrupting the responder's sleep during these 3 days
The times-of-day that notifications were received during these 3 days occurred across a large window, judging by the alert timestamp distribution

This responder's manager is made aware that these first 3 days of the month were untenable, so he rotates a teammate in for an override. The responder goes off-call for health recovery, and the health score shows the remediation trends, as shown below.

3. The manager, team lead, or HR may step in with more aggressive remediation strategies for situations when notification relief cycles are not enough to regain the responder's score in a timely manner. The next best plan of action for remediation is PTO because the health recovery rate is higher than both 1 and 2 above.

4. Other employee incentives or ways to address poor health as a strategy for recovery

5. Operations health information is also used within the context of this invention by providing a manager insight into the trade-offs between the near-term fixes to team health vs. the investment in fixing the reasons for low health, i.e. unhealthy services and parts of the infrastructure which are known problems. If these issues re-occur over time, operations health highlights these trends (both by measuring responder health and service health), which will trigger leaders to address the problem source and fix the issues causing pain for their employees and the overall organization.

6. Responders and team leaders can improve their operations health by using this invention to measurably diagnose health and respond by making use of the appropriate tools that represent operations management best practices, such as:
a. Updating overly sensitive trigger levels in monitoring tools
b. Leveraging event rules to suppress low urgency events so that non-actionable alerts don't become interrupt notifications
c. Employ the use of global routing of events so that the right events go through the appropriate service aligned with the business
d. Turn on alert grouping to cluster associated alerts and reduce overall alert noise, and therefore notification volume
e. Enable notification buffering, which adds a delay on notifications before final transmission, and reduces the amount of transient (flapping) notifications

7. Organizational leaders (SVP's and top senior executives) can also see the impact to the that services with low health have on their business. Business health is derived from a combination of both operational and business metrics, including:
the overall health score for a critical service or feature
revenue lost by that critical service
the service availability, in terms of reliability
the duration of degraded performance of that service or feature
the number of major incidents having to do with the service or feature

Accordingly, in some embodiments, resource management engine may provide interactive reports that enable organizational leadership to discover the incidents that may have the largest impact to the business. This information may be used to prioritize which services or teams receive focus, attention, repair, optimization, or the like. Interactive reports may provide links between each critical service and the teams and responders associated with the service as well as the associated health sub-scores that may be associated with those elements.

### Generalized Operations

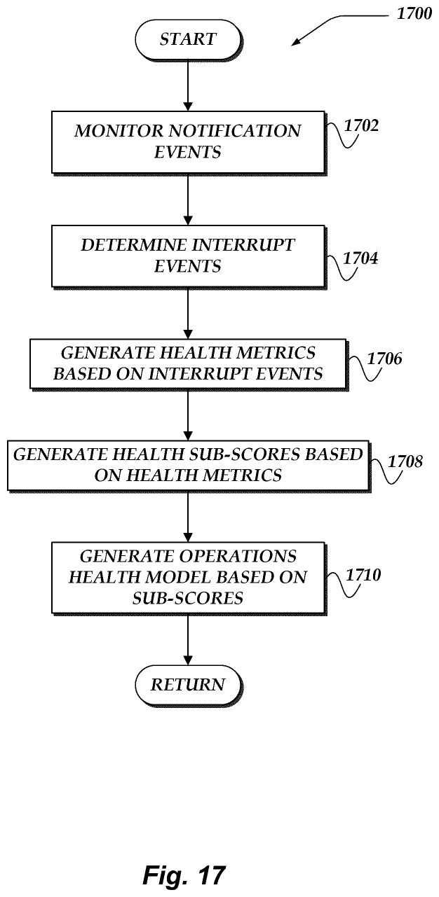

FIGS. 17-20 represent the generalized operations for operations health management in accordance with at least one of the various embodiments. In at least one of the various embodiments, processes 1700, 1800, 1900, and 2000 described in conjunction with FIGS. 17-20 may be implemented by or executed on an operations management server computer, a network computer, or the like, such as, network computer 300 of FIG. 3. In other embodiments, these systems, operations, processes, or portions thereof, may be implemented by or executed on a plurality of network computers, such as network computer 300 of FIG. 3. In yet other embodiments, these systems, operations, processes, or portions thereof, may be implemented by or executed on one or more virtualized computers, such as, those in a cloud-based environment. However, embodiments are not so limited and various combinations of network computers, client computers, or the like may be utilized. Further, in at least one of the various embodiments, some or all of the actions performed by processes 1700, 1800, 1900, and 2000 may be executed in part by ingestion engine 322, modeling engine 324, resource management engine 325, analysis engine 326, or the like, or combination thereof.

FIG. 17 illustrates an overview flowchart for process 1700 for operations health management in accordance with at least one the various embodiments. After a start block, at block 1702, in at least one of the various embodiments, notification events for one or more organizations may be monitored. The particular types or formats of the notification events may vary depending on the type of organization. However, in most cases, notifications events may comprise description information, time stamps, source, destination, severity, status, or the like, or combination thereof, that are associated with one or more operations event or operations incidents that an organization may experience. In one or more of the various embodiments, notification events may correspond to one or more actual notifications provided to one or more responders that may be responsible for the services or application associated with the one or more operations events. For example, notification events may include, email messages, SMS texts, pager pages, telephone calls (live or automated), user-interface alarms, mobile phone push-notifications, or the like, or combination thereof.

In at least one of the various embodiments, notification events may be parsed or processed by the operations management system to determine one or more characteristics, such as, timestamps, source, severity, responsible response, or the like. In at least one of the various embodiments, various metrics that may be associated with the notification events may be determined or collected by the operations management system.

In at least one of the various embodiments, notification events may be associated with specific applications, services, groups, teams, departments, individuals within the same organization. Also, operations events may be associated with particular processes of the organizations.

In at least one of the various embodiments, configuration information may be defined that associates one or more notification events with particular parts of an organization. Also, this configuration information may associate particular notification events to particular persons, projects, tasks, teams, groups, customers, locations, or the like, or combination thereof. In at least one of the various embodiments, the configuration information may include rules/instructions that may be written in one or more programming languages. Further, in at least one of the various embodiments, configuration information may include pattern matching information (e.g., regular expressions) that may be used to determine the associations for notification events.

At block 1704, in at least one of the various embodiments, the operations resource management engine may be arranged to determine one or more interrupt events from among the one or more notification events. In this context, for some embodiments, interrupt events represent notification events that are configured an organization to immediately alert responders and shift their focus away from whatever they are doing. In other words, interrupt events may be those notification events that may be related to critical problems which are intended to be addressed immediately, no matter if the responder is sleeping, or they are having a weekend visits a park with their children, out for dinner, at a holiday party with friends, or the like. Accordingly, in some embodiments, interrupt events may include SMS message, phone calls, push notifications, or the like. For some embodiments, examples of non-interrupt notifications may include informational or reporting emails provided to the user for information purposes only.

At block 1706, in at least one of the various embodiments, a resource management engine may be arranged to generate one or more health metrics based on the interrupt events. In one or more of the various embodiments, as described health metrics may include metrics associated with interrupt events that may be generated by an organization. In some embodiments, metrics may include the rates, counts, averages, time-bucket aggregations, or the like, as described in more detail above.

In some embodiments, one or more metrics may be collected or generated in real-time. Likewise, in some embodiments, one or more metrics may be generated from event log data after the fact.

In one or more of the various embodiments, an ingestion engine, such as, ingestion engine 322, may be arranged to employ information provided by configuration information, rules, user-input, or the like, for determining the specific event characteristics to log. Likewise, in one or more of the various embodiments, a resource management engine or an analysis engine, such as, resource management engine 325, or analysis engine 326 may be arranged to employ information provided by configuration information, rules, user-input, or the like, for determining the specific health metrics to provide.

At block 1708, in one or more of the various embodiments, the operations health system may be arranged to generate one or more health sub-scores based on the collected health metrics models. In one or more of the various embodiments, as described above in more detail, a resource management engine, modeling engine, or analysis engine, such as, resource management engine 325, modeling engine 324, or analysis engine 326 may be arranged to generate one or more health sub-scores based on the collected health metrics.

In one or more of the various embodiments, one or more health sub-score models may be used to generate one or more health sub-scores. In some embodiments, one or more health metric values may be provided to an associated health sub-score model to provide the relevant health sub-scores. For example, in some embodiments, a health sub-score may be defined to represent how far a particular health metric deviates from its corresponding health sub-score model. In contrast, one or more health sub-scores may be generated based on applying statistical methods directly to one or more associated metrics rather than being generated using a model. For example, in one or more of the various embodiments, a health sub-score may be computed by averaging or normalizing raw health metrics rather than using a health sub-score model.

At block 1710, in at least one of the various embodiments, the operations health system may be arranged to generate operations health model the one or more organizations based on the health sub-scores generated in block 1708. As described in greater detail above, the one or more health sub-scores may be provided to an operations health model that provides an operations health score based on the provided health sub-scores. Next, control may be returned to a calling process.

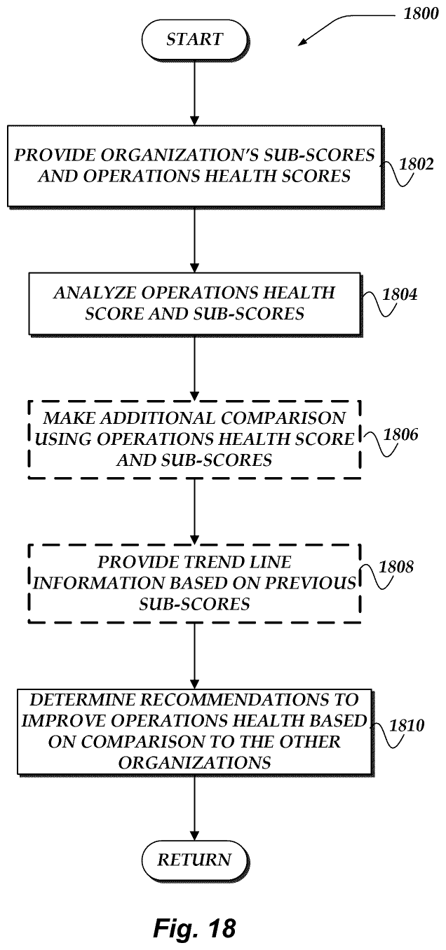

FIG. 18 illustrates a flowchart for process 1800 for operations health management in accordance with at least one the various embodiments. After a start block, at block 1802, in at least one of the various embodiments, the operations health system may be arranged to instantiate one or more resource management engines, modeling engines, analysis engines and provide them health sub-scores and operations health scores for an organization.

At block 1804, in one or more of the various embodiments, the resource management engines may be arranged to analyze the operations health score or the one or more health sub-scores. In one or more of the various embodiments, the analysis may include comparing the operations health scores of the organization to one or more other organizations managed by the operations management system.

In one or more of the various embodiments, the operations health score or the health sub-scores of the organization that is the subject of analysis may be used for one or more additional comparisons. In at least one of the various embodiments, the scores may be used to compare a variety of different dimensions of operations health. As described above in more detail, in at least one of the various embodiments, various types of comparisons may be generated from data-mining the health scores or health metrics for the organizations that are being managed. A few examples are listed below, but one of ordinary skill in the art will appreciate an operations management system may perform other comparisons as well.

In at least one of the various embodiments, the operations health system may process the interrupt events for an organization using various techniques (e.g., data mining, informatics, machine learning, or the like) to identify and perform relevant comparisons.

In one or more of the various embodiments, configuration information that may include rules, instructions, threshold values, conditions, model selection rules, health metric selection rules, normalization rules, or the like, or combination thereof, may be provided for consumption by one or more of the resource management engine, modeling engine, analysis engine, or the like.

In at least one of the various embodiments, the health sub-scores or overall operations health scores for the organization may be compared to the like scores of other organizations. In some embodiments, organizations may be compared to other organizations that are similar in one or more characteristics to each other. In at least one of the various embodiments, comparing organizations that may have some similarities may produce meaningful apples-to-apples comparisons. Accordingly, the organizations may be categorized, segmented, or bucketed, using a variety of criteria, such as, company size, industry, geographic location, number of employees, or the like.

Also, in at least one of the various embodiments, the operations health sub-scores may be used to track the operations health of components of an organization over time. In such cases, the tracked values may be compared to one or more health sub-score models as well as previous health scores of the same organization. The comparisons against the health sub-score models or historical health sub-score values may be used to determine if the operations health for the organization is improving or degrading.

Also, in at least one of the various embodiments, the operations health score or the health sub-scores of the organization may be used to compare different departments, groups, teams, or the like, within the same organization. Accordingly, the comparisons may be used identify one or more departments, groups, teams, or the like, that are operating such that their operations health may be below average or above average.

At block 1808, in at least one of the various embodiments, optionally, previous health sub-score values or the overall operations health score may be used to generate one or more visualization that may be used for illustrating trends or predictions for one or more health sub-scores. In at least one of the various embodiments, various parameters may be set using a user-interface or configuration information to control the time windows, visualization formats (e.g., charts, graphs, scales, and so on) used to generate the visualizations. In some embodiments, the running averages may also be included in one or more visualizations. Accordingly, in one or more of the various embodiments, as shown above, the one or more visualization may be included in one or more interactive reports that enable a user to explore relationships between their organization's health scores or health sub-scores.

At block 1810, in one or more of the various embodiments, the operations health system may be arranged to determine one or more recommendations that may improve operations health. In some embodiments, one or more of the recommendations may be based on comparing the health metrics, health sub-scores, or the like, of the organization with other organizations. In one or more of the various embodiments, historical records associated with other organizations may be used to identify one or more organizations that were experiencing operations health degradation similar to the organization being analyzed. Accordingly, one or more actions that resulted in improvement the operations health of those organizations may be determined and recommended to a user. Next, control may be returned to a calling process.

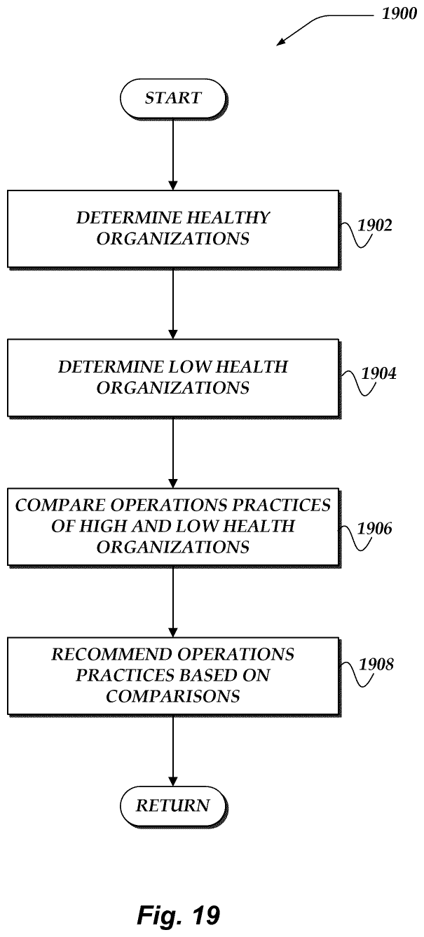

FIG. 19 illustrates a flowchart for process 1900 for operations health management in accordance with at least one the various embodiments. After a start block, at block 1902, in at least one of the various embodiments, the operations health management system may be arranged to determine one or more healthy organizations. In some embodiments, healthy organizations in this context may be determined based on their operations health score exceeding a defined threshold. In some embodiments, the threshold may be an literal score value. Or, in some embodiments, thresholds may be determine based on one or more operations health models, or the like. Also, in some embodiments, threshold for determining healthy organizations may be based on one or more statistical formula/values associated with a set of organizations, such as, mean, median, score distributions (e.g., n-standard deviations), or the like, rather than being limited fixed or literal threshold values as described in more detail above.

At block 1904, in one or more of the various embodiments, the operations health management system may be arranged to determine one or more low health organizations. In one or more of the various embodiments, determining low health organizations may include actions similar to as described for block 1902 except with the goal to identity low health organizations as defined using one or more threshold values, formulas, models, or the like.

At block 1906, in one or more of the various embodiments, the operations health management system may be arranged to compare operations practices of the determined high health and determined low health organizations. In one or more of the various embodiments, a resource management engine or analysis engine may be arranged to have access to configuration information associated with an organization's operations management processes. In some embodiments, this information may include, responder on-call schedules, notification methods, incident escalation rules, event priority settings, or the like. Also, in one or more of the various embodiments, change history associated with operations management may be analyzed as well to identify changes in process that may correlate with increases or decreases in an organizations operations health or health sub-scores.

At block 1908, in one or more of the various embodiments, the operations health management system may be arranged to recommend one or more operations practices or changes to existing operations processes based on the comparisons or analysis described in block 1906. Next, control may be returned to another caller.

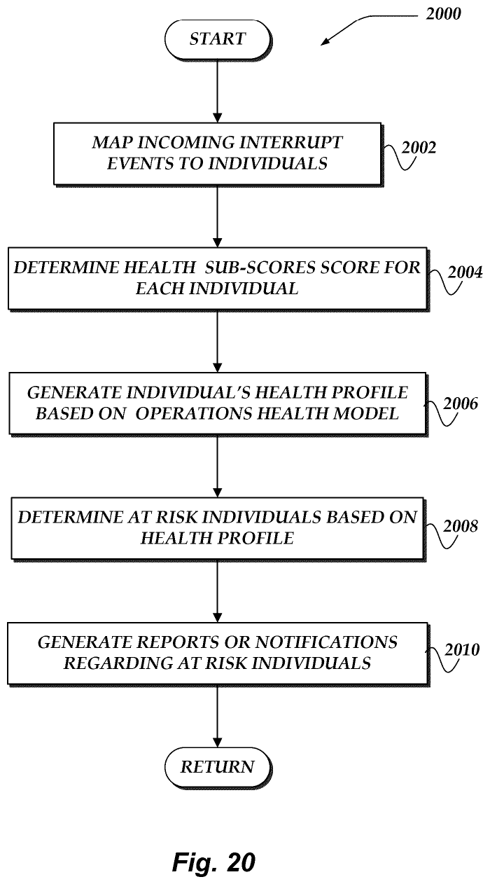

FIG. 20 illustrates a flowchart for process 2000 for generate individual responder health profiles in accordance with at least one the various embodiments. After a start block, at block 2002, in at least one of the various embodiments, an operations health management system may be arranged to map one or more incoming interrupt events to one or more individual responders. In one or more of the various embodiments, there may be one or more responders responsible for each incoming notification event. Accordingly, in some embodiments, a resource management engine may be arranged to provide health metrics that correspond to individual responders. In some embodiments, one or more notification events may be associated with more than one responder. For example, an event may correspond to an incident that affect two or more services or departments of an organization. Thus, in this example, there may be more than one different responders associated with the different services or departments that receive notification for the same operation events or incident.

At block 2004, in one or more of the various embodiments, the operations health management system may be arranged to determine one or more health sub-scores for the one or more responders. In one or more of the various embodiments, the health sub-scores determined for a given responder may be based on the quantity and characteristics of the interrupt events that may be mapped the individual responder. As described above, one or more health sub-score models may be provided health metrics or health sub-scores that are associated with an individual responder to determine health sub-scores that correspond to that individual responder. Likewise, in one or more of the various embodiments, an overall operations health score may be provided for each individual responder by providing their health sub-scores to an operations health model.

At block 2006, in one or more of the various embodiments, the operations health management system may be arranged to generate a health profile for individual responders based on their health sub-scores or their operations health scores. In one or more of the various embodiments, the resource management engine may be arranged to generate health profiles by one or more mapping health related employee/responder actions to their health sub-scores or operations health score. In one or more of the various embodiments, the particular mapping of responder actions to health sub-scores or operations health score may be defined in information provided from configuration information, rules, computer readable instructions, user-input, or the like. For example, one or more responder actions, such as, leaving an organization, taking sick days, productivity measures, or the like, may be correlated with one or more health sub-scores. Accordingly, in one or more of the various embodiments, responder health profiles may provide tracking or report mechanisms that enable organizations to identify which responders are at risk of taking actions that may be detrimental to an organization because of operational pain.

At block 2008, in one or more of the various embodiments, the operations health management system may be arranged to determine one or more at risk responders based on their health profile. In one or more of the various embodiments, a resource management engine may be arranged to monitor health profiles of its responders to identity at-risk individuals. In some embodiments, the monitoring may include comparing health profiles to one or more threshold values, models, or the like, that may be indicative of risk. In some embodiments, the particular risk indication (e.g., threshold values, models, or the like) may be determined using configuration information, rule-based policies, rules, computer readable instructions, user-input, or the like, or combination thereof.

In one or more of the various embodiments, a modeling engine may be arranged to provide on or more risk models that correlate one or more health sub-score or operations health scores to adverse responders actions. Accordingly, in one or more of the various embodiments, the risk models may be employed to predict the potential for adverse responder actions before they occur.

At block 2010, in one or more of the various embodiments, the operations health management system may be arranged to generate one or more reports or notifications regarding the at risk responders. Next, control may be returned to a calling process.

It will be understood that each block of the flowchart illustration, and combinations of blocks in the flowchart illustration, can be implemented by computer program instructions. These program instructions may be provided to a processor to produce a machine, such that the instructions, which execute on the processor, create means for implementing the actions specified in the flowchart block or blocks. The computer program instructions may be executed by a processor to cause a series of operational steps to be performed by the processor to produce a computer-implemented process such that the instructions, which execute on the processor to provide steps for implementing the actions specified in the flowchart block or blocks. The computer program instructions may also cause at least some of the operational steps shown in the blocks of the flowchart to be performed in parallel. Moreover, some of the steps may also be performed across more than one processor, such as might arise in a multi-processor computer system. In addition, one or more blocks or combinations of blocks in the flowchart illustration may also be performed concurrently with other blocks or combinations of blocks, or even in a different sequence than illustrated without departing from the scope or spirit of the invention.

Accordingly, blocks of the flowchart illustration support combinations of means for performing the specified actions, combinations of steps for performing the specified actions and program instruction means for performing the specified actions. It will also be understood that each block of the flowchart illustration, and combinations of blocks in the flowchart illustration, can be implemented by special purpose hardware based systems, which perform the specified actions or steps, or combinations of special purpose hardware and computer instructions. The foregoing example should not be construed as limiting or exhaustive, but rather, an illustrative use case to show an implementation of at least one of the various embodiments of the invention.

Further, in one or more embodiments (not shown in the figures), the logic in the illustrative flowcharts may be executed using an embedded logic hardware device instead of a CPU, such as, an Application Specific Integrated Circuit (ASIC), Field Programmable Gate Array (FPGA), Programmable Array Logic (PAL), or the like, or combination thereof. The embedded logic hardware device may directly execute its embedded logic to perform actions. In at least one embodiment, a microcontroller may be arranged to directly execute its own embedded logic to perform actions and access its own internal memory and its own external Input and Output Interfaces (e.g., hardware pins or wireless transceivers) to perform actions, such as System On a Chip (SOC), or the like.

What is claimed is:

1. An apparatus comprising:
a memory; and
a processor, the processor configured to execute instructions stored in the memory to:
determine interrupt events from a plurality of notification events generated by one or more of a plurality of computer systems, wherein the interrupt events are determined based on respective corresponding notification events being sent in respective manners designed to immediately alert a responder;
receive alert data indicating occurrence of monitored conditions in a managed information technology environment;
assign, based on the alert data, incidents to the responder;
modify, with respect to the responder, a notification configuration based on resolution data associated with the incidents and the interrupt events; and
transmit a notification to the responder according to the modified notification configuration in response to assigning a new incident to the responder.

2. The apparatus of claim 1, wherein to modify, with respect to the responder, the notification configuration based on the resolution data associated with the incidents and the interrupt events comprises to:
determine to modify the notification configuration in response to determining that at least one of mean-time-to-acknowledge of the incidents, mean-time-to-resolve of the incidents, a number of resolved incidents of the incidents, or a number of escalated incidents of the incidents exceed respective baseline values.

3. The apparatus of claim 1, wherein the modified notification configuration indicates to buffer notifications for a threshold duration prior to transmitting notifications to the responder.

4. The apparatus of claim 3, wherein to transmit the notification to the responder according to the modified notification configuration in response to assigning the new incident to the responder comprises to:
assign, based on an incoming alert, the new incident to the responder;
buffer a notification associated with the new incident according to the modified notification configuration; and
determine whether to transmit the notification associated with the new incident to the responder based on a determination of whether the incoming alert is resolved within the threshold duration of assigning the new incident to the responder.

5. The apparatus of claim 1, wherein the processor is further configured to execute instructions stored in the memory to:
modify, with respect to the responder, a configuration based on the resolution data associated with the incidents and the interrupt events wherein the modified configuration indicates to stop assigning new incidents to the responder within a specified duration.

6. The apparatus of claim 1, wherein the modified notification configuration indicates to cluster multiple alerts as a single incident and transmitting a one notification to the responder based on the single incident.

7. The apparatus of claim 1, wherein the respective manners designed to immediately alert the responder comprise at least one of a short message service, a multimedia messaging service, a phone call, a pager page, or a push notification.

8. A method, comprising:
determining interrupt events from a plurality of notification events generated by one or more of a plurality of computer systems, wherein the interrupt events are determined based on respective corresponding notification events being sent in respective manners designed to immediately alert a responder;
receiving alert data indicating occurrence of monitored conditions in a managed information technology environment;
assigning, based on the alert data, incidents to the responder;
modifying, with respect to the responder, a notification configuration based on resolution data associated with the incidents and the interrupt events; and
transmitting a notification to the responder according to the modified notification configuration in response to assigning a new incident to the responder.

9. The method of claim 8, wherein modifying, with respect to the responder, the notification configuration based on the resolution data associated with the incidents and the interrupt events comprises:
determining to modify the notification configuration in response to determining that at least one of mean-time-to-acknowledge of the incidents, mean-time-to-resolve of the incidents, a number of resolved incidents of the incidents, or a number of escalated incidents of the incidents exceed respective baseline values.

10. The method of claim 8, wherein the modified notification configuration indicates to buffer notifications for a threshold duration prior to transmitting notifications to the responder.

11. The method of claim 10, wherein transmitting the notification to the responder according to the modified notification configuration in response to assigning the new incident to the responder comprises:
assigning, based on an incoming alert, the new incident to the responder;
buffering a notification associated with the new incident according to the modified notification configuration; and
determining whether to transmit the notification associated with the new incident to the responder based on a determination of whether the incoming alert is resolved within the threshold duration of assigning the new incident to the responder.

12. The method of claim 8, further comprising:
modifying, with respect to the responder, a configuration based on the resolution data associated with the incidents and the interrupt events wherein the modified configuration indicates to stop assigning new incidents to the responder within a specified duration.

13. The method of claim 8, wherein the modified notification configuration indicates to cluster multiple alerts as a single incident and transmitting a one notification to the responder based on the single incident.

14. The method of claim 8, wherein the respective manners designed to immediately alert the responder comprise at least one of a short message service, a multimedia messaging service, a phone call, a pager page, or a push notification.

15. A non-transitory computer readable medium storing instructions operable to cause one or more processors to perform operations comprising, comprising:
determining interrupt events from a plurality of notification events generated by one or more of a plurality of computer systems, wherein the interrupt events are determined based on respective corresponding notification events being sent in respective manners designed to immediately alert a responder;
receiving alert data indicating occurrence of monitored conditions in a managed information technology environment;
assigning, based on the alert data, incidents to the responder;
modifying, with respect to the responder, a notification configuration based on resolution data associated with the incidents and the interrupt events; and
transmitting a notification to the responder according to the modified notification configuration in response to assigning a new incident to the responder.

16. The non-transitory computer readable medium of claim 15, wherein modifying, with respect to the responder, the notification configuration based on the resolution data associated with the incidents and the interrupt events comprises:
determining to modify the notification configuration in response to determining that at least one of mean-time-to-acknowledge of the incidents, mean-time-to-resolve of the incidents, a number of resolved incidents of the incidents, or a number of escalated incidents of the incidents exceed respective baseline values.

17. The non-transitory computer readable medium of claim 15, wherein the modified notification configuration indicates to buffer notifications for a threshold duration prior to transmitting notifications to the responder.

18. The non-transitory computer readable medium of claim 17, wherein transmitting the notification to the responder according to the modified notification configuration in response to assigning the new incident to the responder comprises:
assigning, based on an incoming alert, the new incident to the responder;
buffering a notification associated with the new incident according to the modified notification configuration; and
determining whether to transmit the notification associated with the new incident to the responder based on a determination of whether the incoming alert is resolved within the threshold duration of assigning the new incident to the responder.

19. The non-transitory computer readable medium of claim 15, wherein the operations further comprise:
modifying, with respect to the responder, a configuration based on the resolution data associated with the incidents and the interrupt events wherein the modified configuration indicates to stop assigning new incidents to the responder within a specified duration.

20. The non-transitory computer readable medium of claim 15, wherein the modified notification configuration indicates to cluster multiple alerts as a single incident and transmitting a one notification to the responder based on the single incident.
